(19) United States
(12) Patent Application Publication
(10) Pub. No.: US 2024/0327915 A1
(43) Pub. Date: Oct. 3, 2024

(54) piRNA BIOMARKER USED FOR DIFFERENTIAL DIAGNOSIS OF PATIENTS WITH DIFFERENT SYMPTOMS AND DISEASE STATUS OF PARKINSON'S DISEASE

(71) Applicants: National Taiwan University, Taipei (TW); National Taiwan University Hospital, Taipei (TW)

(72) Inventors: Shau-Ping LIN, Taipei (TW); Ruey-Meei WU, Taipei (TW); Pin-Jui KUNG, Taipei (TW); Ming-Che KUO, Taipei (TW); Yi-Tzang TSAI, Taipei (TW)

(21) Appl. No.: 18/621,744

(22) Filed: Mar. 29, 2024

Related U.S. Application Data
(60) Provisional application No. 63/455,952, filed on Mar. 30, 2023.

(51) Int. Cl. C12Q 1/6883

(52) U.S. Cl. CPC C12Q 1/6883; C12Q 2600/112; C12Q 2600/158; C12Q 2600/178

(57) ABSTRACT

The present invention provides a biomarker for the differential diagnosis of patients with Parkinson's disease. By using piRNAs as biomarkers, patients with Parkinson's disease with and without mild cognitive impairment, and Parkinson's disease with dementia can be classified early, thereby meeting the needs of improving treatment efficiency and improving the differential diagnosis of patients with atypical Parkinson's disease in the current medical field. Another aspect of the invention provides a kit of biomarkers, therapeutic targets, and a method of detecting whether an individual has Parkinson's disease.

### FIELD OF TECHNOLOGY

The present invention relates to a biomarker for differential diagnosis of a patient with Parkinson's disease, and in particular to application of piRNA in differential diagnosis of a patient with Parkinson's disease; however, the present invention is not limited to this.

### BACKGROUND

Parkinson's disease (PD) is a progressive, age-related, incurable, and debilitating neurodegenerative disease that is second most common in human, which affects more than 1-2% of global population over 65 years old and is second only to Alzheimer's disease (AD).

PD not only causes motor symptoms, but also may be accompanied by cognitive impairment and dementia. Specifically, a patient with Parkinson's disease is often accompanied by motor symptoms (MS), such as slow movement, frozen gait, etc.; or non-motor symptoms (NMS), such as fatigue, sleep disorders, depression, anxiety and the like symptoms.

Among many Parkinson's disease patients with non-motor symptoms on top of motor symptoms, the complication of neurocognitive disorder (NCD) are further divided into patients with PD with mild cognitive impairment (PD-MCI) and patients with PD with dementia (PDD). As PDD patients response differently to medication compared to PD patients without cognitive problem (PDND; Parkinson's Disease, no dementia), it is important to classify PD patients with (PD-MIC and PDD) or without (PDND) cognitive deficits.

Current diagnostic modes used for diagnosing PD-MCI and PDD patients include: magnetic resonance imaging (MRI), fluorinated deoxyglucose positron emission tomography (FDG-PET) or CSF-, α-Syn, tau test kits, etc.

### SUMMARY

The Summary of the present invention is intended to provide a simplified inventive content of the present invention so that readers can have a basic understanding of the present invention. The Summary of the present invention is not a complete overview of the present invention, and it is not intended to point out important/key elements of embodiments of the present invention or define the scope of the present invention.

Based on the aforementioned content, the inventor believes that the previous practice has shortcomings such as being time-consuming and labor-intensive, having low sensitivity, and being easily limited by the professionalism of testing personnel and testing facilities; and the inventor has even found that about 40% of patients with Parkinson's disease in Taiwan meet the criteria for PD-MCI. Among them, 10% of PD-MCI patients evolve into PDD patients in the early stage, and 80% of PD-MCI patients evolve into PDD patients in the late stage. Therefore, the inventor of the present invention believe that it is necessary to establish a more effective and economical manner to detect, diagnose and differentiate patients with Parkinson's disease with different symptoms at an early stage, and to provide them with effective drug treatment as early as possible.

PIWI-interacting RNA (piRNA) is a small noncoding RNA with a length of approximately 24 to 34 nucleotides, which can bind to PIWI proteins to form PIWI-piRNA complexes. piRNA is mainly expressed in germ cells and inhibits the expression of transposons. In somatic cells, piRNA can act as an endogenous small-interfering RNA (endo-siRNA). piRNA has been found to be associated with a variety of human diseases such as cancer, metabolic diseases, and neurological diseases, etc., and is therefore considered to be useful for identifying drug targets and developing new therapeutic approaches. In view of this, the inventor of the present application have further studied application of piRNA for Parkinson's disease and screened out a series of biomarkers for detecting Parkinson's disease. By these novel biomarkers for Parkinson's disease, it is possible to improve current medical clinical needs for the differential diagnosis of such atypical Parkinson's disease.

Accordingly, one aspect of the present invention provides a biomarker for differential diagnosis of a patient with Parkinson's disease, wherein the biomarker is piRNA, and is derived from plasma or from plasma and brain, or the biomarker is targeting brain related genes.

In one aspect of the present invention, the biomarker is derived from plasma, including all detectable piRNAs-like sequences categorized by their host transcript, including the following (Group 1): RNU2-51P, RNU6-727P, LDOC1, RNU2-16P, EYS, RNU6-37P, RNU2-60P, ENSG00000257657, U6, RNU6-3P, H3-3A, CCDC102B, RNU6-39P, RN7SL767P, FAM53A, PCDH11X, RNU2-26P, IFT88, RNU2-7P, INO80, KIF1B, RNU6-1225P, KTNI, HDACI, UHRFIBPI, ANKHDI, ANKHDI-EIF4EBP3, RN7SL151P, ENSG00000285641, RAB13, RN7SL128P, VKORC1L1, EGR3, RNU1-2, RNY4P7, ENSG00000269707, POU3F3, WDR33, CROCC, Y_RNA, RN7SL87P, MEGF9, PGAPI, RNY4P25, PPHLN1, HS3ST4, SP3, RN7SL597P, RNU6-10P, TMOD3, GFM2, RNU6-658P, MAPT, FOXC1, CNTN3, SNORD57, CDK6, GPALPP1, RN7SL49P, AGBL5, H4C5, RN7SL4P, ZC3HAV1, RNU6-140P, EPB41, EIF3J, RUVBL1, RNU2-37P, RN7SL181P, ENSG00000288684, TOPORS, ULK4, ST6GAL2, ATP13A3, GRAP2, TRAK2, PIP4P1, RNU2-64P, ZNF385D, DUSPI, STAT5B, RGPD5, RNU6-743P, SNORD99, SNHG12, NBPF20, MANF, VTRNA1-1 or TBC1D14.

In one aspect of the present invention, the biomarker is derived from plasma and brain, including all detectable piRNAs-like sequences categorized by their host transcript, including the following (Group 2): LINC00910, RNU2-4P, CTSD, CCDC102B, MAPT, SEC14L1, PGAP1, RN7SL151P, PPHLN1, Y_RNA, C16orf72, ZC3HAVI, RNVU1-7, RN7SL767P, KLHL20, SGTA, DUSP3, KLF2, NUAK1, ACTR3, RAPIGDSI, ZFP36L2, PDXK, PTPRG, MN1, TMOD3, DPP10, TNRC6A, MARCHF8, CLIP4, CYBRD1, PPP3CA, POU3F2, FMNL3, GPATCH8, IQCH, USP7, RAB5C, STRBP, LARGE1, AKAP9, SHARPIN, NFIX, TOPORS, VKORC1L1, GPALPP1, LDB1, KIF21A, ST6GAL2, PARP1, ESRRG, PRRC2C, AFG1L, MBNL3, ERI3, MEGF9, SEMASA, RNU1-2, SNORD17, SPECC1, AKAP8L, ALKBH5, KIF2A, CHKB, GIGYF2, GNG12-ASI, CD99P1, COMMD3-BMI1, ANO6, ATP13A3, HLA-B, DENND4C, GNBI, TMA7, NCOR2, UBAC2, SHANK2, RPL13A, HS3ST4, NOTCH2, CHKB-CPT1B, SEC31A, TRIO, CSDE1, NAP1L1, HSP90B1, PPP1CC, RNF213, ZNF592, RN7SL49P, TMEM185A, MARCKS, LRPAP1, BCL2L1, MACF1, CWC27, BNIP3L, CATSPER2, FBXO7, HAUS7, HDAC1, HDAC8, KTN1, RN7SL1, RNU2-36P, SLC24A3, TPT1 or TREX2.

In one aspect of the present invention, the biomarker is derived from plasma, including all detectable piRNAs-like sequences categorized by their host transcript, including the following (Group 3): ZC3HAV1, CDK6, VKORC1L1, RAB13, MEGF9, RNU6-10P, RNU2-51P, U2, FYB1, UNC13C, THSD4, ZYX, CLCN3, PGAP1, SNX6, RAB13, GCNT1, Y_RNA, M6PR, VPS13D, TNFRSF1A, PAPOLA, EFS, AGO2, ATE1, AFM, IL10RA, ENO2, EXOC2, GCA, ITGB1BP1, ATAD2B, ACHE, INSIG2, GNG11, TUBGCP2, AHNAK, NT5E, GDF11, TTYH3, TANK, ATF7IP, CORO1C, LPCAT3, CERT1, ARL1, ATP5F1B, KCNH2, PIK3R3, LUC7L2, STAM2, AGPS, HERC5, PDCD6, ENPEP, RBM27, PIP5K1B, BLVRA, TCIRG1, SLC18A1, RBM6, ETNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIPI2, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, VWC2L, POC1B, H2BC4, GPHN, ANO1, CDHR3, ZFYVE1, PTPN6, ETS1, HTR1F, TAGLN2, ZBTB38, ZNF513, DTX2, CUL1, SCMH1, BTNL9, COA5, EEF1G, CABCOCO1, MRTO4, CD8B, SLC6A17, CEP63, WSCD2, TSKU, TESK1, DHX15, FGD3, ZNF385A, SLA, TMEM167B, CPM, FBXL14, RIMS2, ADIPOR1, ATP2B4, NHSL1, OAS2, CCDC18, NKX2-3, LPAR6, ATG16L1, SCEL, C7orf25, SPATA16, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, PIK3CG, LRP10, SLC39A10, RPL37A, TIAM2, UBAP2, NCKAP1, CLSTN1, RNU5B-1, RNU6-1309P, RYR2, LGALS8, DNAH14, TMEM181, MDM4, TOR1AIP2, RABGAP1L, IFNGR1, HBS1L, TMCO1, ARHGAP30, SELENBP1, PDSS2, PNISR, CD58, DUSP5, SUFU, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, API5, B3GALT6, SLC7A1, VGLL3, RNU6-629P, SNORD26, RNU6-43P, SNORD64, IGKV3-15, CCDC50, NUDT14, ARHGAP9, SYNE2, LBH, RASGEF1A, DDHD2, GNAT3, RPS6KA2, KMT5B, FAT3, MXDI, RNU2-29P, RNU2-59P, CROCCP2, NBEAL2, KBTBD8, NEBL, ISY1-RAB43, ACTG1P19, HTATIP2, THEM7P, ATF7, BSN-DT, TMEM132D, LINC-PINT, INO80D, LINC00993, MTCO1P51, SOX5, AHCYP2, IRAK4, LINC01807, LRRC23, ANXA2P2, FHIP1A, ABTB2, TRABD2B, USP46, TAX1BP1-AS1, TRGV9, COL3A1, POTEI, HRAT17, ARHGAP25, CFLAR, RNU7-1, SNORD121B, RNU7-19P, NDUFA5, CAPI, TRA2B, EIF3B, CTBP2, GNA12, ADGRL2, TP53BP1, CYB5R4, FLNB, AOAH, ENPP7P4, UBE4B, RHOBTB1, RN7SL242P, LCP1, HNRNPA2B1, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, PABPC1, SLC45A4, CLU, SOX6, KMT2A, E2F8, XKR6, GALNT4, SF3B2, DLG2, GLYATL2, SNHG1, LINC02376, CUL3, C12orf57, FMC1-LUC7L2, POC1B-GALNT4, LINC02416, SNHG14, ACTN1, LINC00639, CCDC85C, MARK3, LINC02533, ATF7-NPFF, H4C1, WASF2, PWAR5, PLCL1, MAMDC2-AS1, SYNGAP1-AS1, RYR3, LINC00862, PHGDH, LINC02752, LINC01117, JAK1, PRKN, GSK3B, DDX54, LONRF2, Metazoa_SRP, PRDM10, RARB, CADM2, CMC1, EIF3A, FRMD4A, LINC01170, LINC02540, MIPOL1, NIBAN1, PPHLN1, PPTC7, RNU6-1062P, RNU6-84P, SNORD6, SORCS3, TAFID, VWF or YY1.

In one aspect of the present invention, the brain-derived piRNA biomarker candidates targeting transcripts expressed in the brain, categorized by the targeted gene name as the following (Group 4): NRSN1, APPL2, TNFAIP8, ATP11A, LINC00847, CASP10, SNX16, MTA3, GRAPL, SMARCA2, LYPD3, ZNF252P-AS1, IRF2, TM9SF2, UNC79, LDC1P, GPR180, EFCAB5, PARP16, SH3D19, VPS37A, THAP8, IQSEC2, PTPRD, WDR7, INO80D, POLR1B, CCDC85C, CPE, STK24, DAAM2-AS1, CTSZ, DNAJC9, HSDL2-AS1, CBR4, IDI2-AS1, ACO2, ADNP-AS1, DIP2C-AS1, LINC00624, MAX, SRGAP2-AS1, NMT1, KLC4, KMT2E, LINC00506, ZNF565, ERAP1, TICAM2-AS1, PIGH, UFLI-AS1, VIM-AS1, KCNQ1OT1, TCHP, RB1, LIX1L-AS1, KCNMA1-AS3, PIGC, CYB5D2, FAM184B, FAR2, CRLF3, VCAN-AS1, STRADB, FTX, SKP2, RBFOX1, RPTOR, GNG12-AS1, CCDC85C, IDI2-AS1, NRSN1, TNFAIP8, KIF27, MEF2A, MT-ND3, NTAQ1, SMU1, SPINT2, PREPL, YJU2, MT-CO3 or THSD1.

In one aspect of the present invention, the biomarker is used for distinguishing patients with non-Parkinson's disease (HC) from patients with Parkinson's disease (PD).

In one aspect of the present invention, the biomarker is RNU2-51P, RNU2-16P, POU3F3, WDR33, CROCC, PGAP1, MAPT or ATP13A3.

In one aspect of the present invention, the biomarker is used for distinguishing HC patients from PD patients, and the PD patients are patients with PD with no dementia (PDND), PD with mild cognitive impairment (PD-MCI) or PD with dementia (PDD); where the biomarker is LINC00910, RNU2-4P, CTSD, CCDC102B, MAPT, SEC14L1, PGAP1, RN7SL151P, PPHLN1, Y_RNA, C16orf72, ZC3HAV1, RNVU1-7, RN7SL767P, KLHL20, SGTA, DUSP3, KLF2, NUAK1, ACTR3, RAPIGDSI, ZFP36L2, PDXK, PTPRG, MN1, TMOD3, DPP10, TNRC6A, MARCHF8, CLIP4, CYBRD1, PPP3CA, POU3F2, FMNL3, GPATCH8, IQCH, USP7, RAB5C, STRBP, LARGE1, AKAP9, SHARPIN, NFIX, TOPORS, VKORC1L1, GPALPP1, LDB1, KIF21A, ST6GAL2, PARP1, ESRRG, PRRC2C, AFG1L, MBNL3, ER13, MEGF9, SEMASA, RNU1-2, SNORD17, SPECC1, AKAP8L, ALKBH5, KIF2A, CHKB, GIGYF2, GNG12-AS1, CD99P1, COMMD3-BMI1, ANO6, ATP13A3, HLA-B, DENND4C, GNB1, TMA7, NCOR2, UBAC2, SHANK2, RPL13A, HS3ST4, NOTCH2, CHKB-CPTIB, SEC31A, TRIO, CSDEI, NAP1L1, HSP90B1, PPP1CC, RNF213, ZNF592, RN7SL49P, TMEM185A, MARCKS, LRPAP1, BCL2L1, MACF1 or CWC27.

In one aspect of the present invention, the biomarker is used for distinguishing HC and PD patients, and the PD patients are PDND or PDD patients; wherein the biomarker is LINC00910, RNU2-4P, RN7SL1, RNU2-36P, HDAC8, TREX2, HAUS7, HDAC1, RN7SL151P, SLC24A3, ZC3HAV1, MAPT, FBX07, TMOD3, CATSPER2, PGAP1, CCDC102B, TOPORS, VKORC1L1, GPALPP1, MEGF9, TPT1, RNVU1-7, RNU1-2, Y_RNA, KTN1, RN7SL767P, BNIP3L, RN7SL49P, TMEM185A, ATP13A3 or BCL2L1.

In one aspect of the present invention, the biomarker is LINC00910, PGAP1, MAPT or ATP13A3.

In one aspect of the present invention, the biomarker is used for distinguishing HC and PD patients, and the PD patients are PDND patients; wherein the biomarker is ZC3HAV1, CDK6, VKORC1L1, RAB13, MEGF9, RNU6-10P, RNU2-51P, U2 or FYB1.

In one aspect of the present invention, the biomarker is used for distinguishing HC and PD patients, and the PD patients are PD-MCI patients; wherein the biomarker is UNC13C, THSD4, ZYX, CLCN3, PGAP1, SNX6, RAB13, MEGF9, GCNT1 or Y_RNA.

In one aspect of the present invention, the biomarker is used for distinguishing HC and PD patients, and the PD patients are PDD patients; wherein the biomarker is M6PR, VPS13D, TNFRSF1A, PAPOLA, EFS, AGO2, ATE1, AFM, IL10RA, ENO2, EXOC2, GCA, ITGB1BP1, ATAD2B, ACHE, INSIG2, GNG11, TUBGCP2, AHNAK, NT5E, GDF11, TTYH3, TANK, ATF7IP, CORO1C, LPCAT3, CERT1, ARL1, ATP5F1B, KCNH2, PIK3R3, LUC7L2, STAM2, AGPS, HERC5, PDCD6, ENPEP, RBM27, PIP5K1B, BLVRA, TCIRG1, SLC18A1, RBM6, ETNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIPI2, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, VWC2L, POC1B, H2BC4, GPHN, ANO1, CDHR3, ZFYVE1, PTPN6, ETS1, HTR1F, TAGLN2, ZBTB38, ZNF513, DTX2, CUL1, SCMH1, BTNL9, COA5, EEF1G, CABCOCO1, MRTO4, CD8B, SLC6A17, CEP63, WSCD2, TSKU, TESK1, DHX15, FGD3, ZNF385A, SLA, TMEM167B, CPM, FBXL14, RIMS2, ADIPOR1, ATP2B4, NHSL1, OAS2, CCDC18, NKX2-3, LPAR6, ATG16L1, SCEL, C7orf25, SPATA16, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, PIK3CG, LRP10, SLC39A10, RPL37A, TIAM2, UBAP2, NCKAP1, CLSTN1, Y_RNA, RNU5B-1, RNU6-1309P, RYR2, LGALS8, DNAH14, TMEM181, MDM4, TOR1AIP2, RABGAP1L, IFNGR1, HBS1L, TMCO1, ARHGAP30, SELENBP1, PDSS2, PNISR, CD58, DUSP5, SUFU, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, API5, B3GALT6, SLC7A1, VGLL3, RNU6-629P, SNORD26, RNU6-43P, SNORD64, IGKV3-15, CCDC50, NUDT14, ARHGAP9, SYNE2, LBH, RASGEF1A, DDHD2, GNAT3, RPS6KA2, KMT5B, FAT3, MXD1, RNU2-29P, RNU2-59P, CROCCP2, NBEAL2, KBTBD8, NEBL, ISY1-RAB43, ACTG1P19, HTATIP2, THEM7P, ATF7, BSN-DT, TMEM132D, LINC-PINT, INO80D, LINC00993, MTCO1P51, SOX5, AHCYP2, IRAK4, LINC01807, LRRC23, ANXA2P2, FHIP1A, ABTB2, TRABD2B, USP46, TAX1BP1-AS1, TRGV9, COL3A1, POTEI, HRAT17, ARHGAP25, CFLAR, RNU7-1, SNORD121B, RNU7-19P, NDUFA5, CAP1, TRA2B, EIF3B, CTBP2, GNAI2, ADGRL2, TP53BP1, CYB5R4, FLNB, AOAH, ENPP7P4, UBE4B, RHOBTB1, RN7SL242P, LCP1, HNRNPA2B1, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, PABPC1, SLC45A4, CLU, SOX6, KMT2A, E2F8, XKR6, GALNT4, SF3B2, DLG2, GLYATL2, SNHG1, LINC02376, CUL3, C12orf57, FMC1-LUC7L2, POC1B-GALNT4, LINC02416, SNHG14, ACTN1, LINC00639, CCDC85C, MARK3, LINC02533, ATF7-NPFF, H4C1, WASF2, PWAR5, PLCL1, MAMDC2-AS1, SYNGAP1-AS1, RYR3, LINC00862, PHGDH, LINC02752, LINC01117, JAK1, PRKN or GSK3B.

In one aspect of the present invention, the biomarker is used for distinguishing PDND and PD-MCI patients, wherein the biomarker is UNC13C, PRDM10, DDX54, ZYX, CLCN3, SNX6, Y_RNA, GCNT1, RARB, LONRF2, RNU2-51P, SNHG14 or Metazoa_SRP.

In one aspect of the present invention, the biomarker is used for distinguishing PDND and PDD patients; wherein the biomarker is M6PR, VPS13D, TNFRSF1A, PAPOLA, EFS, AGO2, ATE1, AFM, IL10RA, ENO2, EXOC2, GCA, ITGB1BP1, ATAD2B, ACHE, INSIG2, GNG11, TUBGCP2, AHNAK, NT5E, GDF11, TTYH3, TANK, ATF7IP, CORO1C, VWF, LPCAT3, CERT1, ARL1, ATP5F1B, KCNH2, YY1, PIK3R3, STAM2, AGPS, PDCD6, ENPEP, RBM27, PIP5K1B, BLVRA, TCIRG1, SLC18A1, RBM6, ETNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIPI2, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, WC2L, POC1B, PPHLN1, CDHR3, PABPC1, ZFYVE1, PTPN6, ETS1, HTR1F, TAGLN2, ZBTB38, ZNF513, TAF1D, DTX2, CUL1, RABGAP1L, SCMH1, MIPOL1, BTNL9, COA5, EEF1G, CABCOCO1, MRTO4, CD8B, SLC6A17, CEP63, WSCD2, TSKU, CMC1, TESK1, DHX15, FGD3, ZNF385A, SLA, TMEM167B, CPM, FBXL14, RIMS2, ADIPOR1, ATP2B4, NHSL1, OAS2, CCDC18, NKX2-3, LPAR6, ATG16L1, SCEL, C7orf25, PPTC7, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, LRP10, SLC39A10, RPL37A, TIAM2, UBAP2, EIF3B, NCKAP1, CLSTN1, Y_RNA, RNU5B-1, RNU6-1309P, SNORD6, RYR2, LGALS8, DNAH14, PRKN, TMEM181, MDM4, TOR1AIP2, IFNGR1, HBS1L, TMCO1, ARHGAP30, SELENBP1, PDSS2, EIF3A, PNISR, CD58, DUSP5, SORCS3, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, API5, B3GALT6, SLC7A1, VGLL3, CADM2, RNU6-629P, SNORD26, SNORD64, RYR3, IGKV3-15, CCDC50, NUDT14, ARHGAP9, SYNE2, LBH, RASGEF1A, DDHD2, GNAT3, CLU, RPS6KA2, KMT5B, FAT3, LINC00993, MTCO1P51, AHCYP2, IRAK4, LINC01807, LRRC23, FHIP1A, ABTB2, TRABD2B, TAX1BP1-AS1, TRGV9, COL3A1, POTEI, HRAT17, LINC02540, ARHGAP25, CFLAR, SNORD121B, RNU7-19P, GPHN, NDUFA5, TRA2B, PIK3CG, CTBP2, GNAI2, ADGRL2, FRMD4A, TP53BP1, CYB5R4, FLNB, AOAH, ENPP7P4, UBE4B, RHOBTB1, RN7SL242P, LCP1, HNRNPA2B1, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, SLC45A4, SOX6, DLG2, KMT2A, E2F8, XKR6, GALNT4, GLYATL2, SNHG1, LINC02376, CUL3, SOX5, POC1B-GALNT4, LINC02416, SNHG14, ACTN1, LINC00639, CCDC85C, MARK3, LINC02533, ATF7-NPFF, RNU6-84P, H4C1, WASF2, PWAR5, PLCL1, MAMDC2-AS1, LINC01170, SYNGAP1-AS1, LINC00862, PHGDH, LINC02752, LINC01117 or JAK1.

In one aspect of the present invention, the biomarker is used for distinguishing PD-MCI and PDD patients; wherein the biomarker is M6PR, VPS13D, TNFRSF1A, PAPOLA, EFS, AGO2, ATE1, AFM, ENO2, EXOC2, GCA, ITGB1BP1, ATAD2B, ACHE, INSIG2, GNG11, TUBGCP2, NT5E, TTYH3, TANK, ATF7IP, CORO1C, LPCAT3, CERT1, ARL1, ATP5F1B, KCNH2, YY1, PIK3R3, LUC7L2, STAM2, AGPS, HERC5, PDCD6, ENPEP, RBM27, PIP5K1B, BLVRA, TCIRG1, SLC18A1, RBM6, TNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIPI2, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, VWC2L, H2BC4, ANO1, PHLN1, CDHR3, ZFYVE1, PTPN6, HTR1F, TAGLN2, ZBTB38, ZNF513, DTX2, CUL1, RABGAP1L, SCMH1, MIPOL1, BTNL9, COA5, EEF1G, CABCOCO1, MRTO4, CD8B, SLC6A17, CEP63, WSCD2, TSKU, TESK1, DHX15, FGD3, ZNF385A, TMEM167B, CPM, RIMS2, ADIPOR1, ATP2B4, HSL1, OAS2, CCDC18, NKX2-3, LPAR6, ATG16L1, SCEL, C7orf25, SPATA16, PPTC7, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, PIK3CG, LRP10, SLC39A10, RPL37A, UBAP2, NCKAP1, CLSTN1, Y_RNA, RNUSB-1, RNU6-1309P, RYR2, LGALS8, DNAH14, TMEM181, MDM4, TOR1AIP2, IFNGR1, HBS1L, TMCO1, SELENBP1, PDSS2, EIF3A, PNISR, CD58, DUSP5, SORCS3, SUFU, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, API5, B3GALT6, SLC7A1, VGLL3, CADM2, RNU6-1062P, RNU6-629P, SNORD26, RNU6-43P, SNORD64, RYR3, IGKV3-15, CCDC50, NUDT14, ARHGAP9, SYNE2, LBH, RASGEF1A, DDHD2, GNAT3, CLU, RPS6KA2, KMT5B, ARHGAP25, FAT3, MXD1, RNU2-29P, RNU2-59P, CROCCP2, NBEAL2, KBTBD8, ISY1-RAB43, ACTG1P19, HTATIP2, THEM7P, ATF7, BSN-DT, TMEM132D, LINC-PINT, INO80D, LINC00993, MTCO1P51, SOX5, AHCYP2, IRAK4, LINC01807, LRRC23, ANXA2P2, FHIP1A, ABTB2, TRABD2B, TAX1BP1-AS1, TRGV9, POTEI, CUL3, HRAT17, LINC02540, CFLAR, RNU7-1, SNORD121B, RNU7-19P, NDUFA5, CAP1, TRA2B, EIF3B, CTBP2, GNAI2, ADGRL2, FRMD4A, TP53BP1, CYB5R4, AOAH, ENPP7P4, UBE4B, RHOBTB1, RN7SL242P, HNRNPA2BI, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, PABPC1, SLA, SLC45A4, SOX6, IL10RA, DLG2, KMT2A, E2F8, XKR6, GLYATL2, SNHG1, LINC02376, C12orf57, FMC1-LUC7L2, GPHN, LINC02416, SNHG14, ACTN1, LINC00639, CCDC85C, MARK3, LINC02533, ATF7-NPFF, RNU6-84P, H4C1, WASF2, PWAR5, PLCL1, MAMDC2-AS1, LINC01170, SYNGAP1-AS1, LINC00862, PHGDH, LINC02752, LINC01117 or PRKN.

In one aspect of the present invention, the biomarker is RNU2-51P or PGAP1.

In one aspect of the present invention, the biomarker is used for distinguishing HC and PD patients, and the PD patients are PDND patients; wherein the biomarker is NRSN1, APPL2, TNFAIP8, ATP11A, LINC00847, CASP10, SNX16, MTA3, GRAPL, SMARCA2, LYPD3, ZNF252P-AS1, IRF2, TM9SF2, UNC79, LDC1P, GPR180, EFCAB5, PARP16, SH3D19, VPS37A, THAP8, IQSEC2, PTPRD, WDR7, INO80D, POLR1B, CCDC85C, CPE, STK24, DAAM2-AS1, CTSZ, DNAJC9, HSDL2-AS1, CBR4, IDI2-AS1, ACO2, ADNP-AS1, DIP2C-AS1, LINC00624, MAX, SRGAP2-AS1, NMT1, KLC4, KMT2E, LINC00506, ZNF565, ERAP1, TICAM2-AS1, PIGH, UFL1-AS1, VIM-AS1, KCNQ1OT1, TCHP, RB1, LIX1L-AS1, KCNMA1-AS3, PIGC, CYB5D2, FAM184B, FAR2, CRLF3, VCAN-AS1, STRADB, FTX, SKP2, RBFOX1, RPTOR or GNG12-AS1.

In one aspect of the present invention, the biomarker is used for distinguishing HC and PD patients, and the PD patients are PDD patients; wherein the biomarker is NRSN1, SGMS1-AS1, IRF2, LINC00847, SMARCA2, WDR87BP, LYPD3, ATP11A, APPL2, TM9SF2, GPR180, PREPL, YJU2, TNFAIP8, MT-CO3, FAT3, MTCO3P12, SH3D19, MT-TH, IQSEC2, UNC79, CCDC85C, LBHDI, SLC5A2, ARG2, GPR156, AUNIP, FOXM1, IDI2-AS1, TMEM165 or CIQTNF7-AS1.

In one aspect of the present invention, the biomarker is used for distinguishing PDND and PDD patients; wherein the biomarker is NTAQ1, SPINT2, KIF27, THSD1, SMU1, MT-ND3 or MEF2A.

In one aspect of the present invention, the biomarker is MT-CO3, SH3D19, TM9SF2, LYPD3, LINC00847, PREPL, YJU2, GPR180, IRF2, IQSEC2, ATP11A, GRAPL, CASP10, UNC79, SMARCA2, ZNF252P-AS1 or MTA3.

In one aspect of the present invention, the piRNA targets chromosome 1, 5, 7, 14, 15, 16, 17, 18 or 19 of the brain.

In one aspect of the present invention, the piRNA targets chromosome 2, 6, 9, 11, 13, 15, 16 or 17 of the brain.

Another aspect of the present invention provides a method for detecting whether an individual has Parkinson's disease, comprising the following steps: I. acquiring a specimen from the individual; II. separating a target detection object from the specimen; III. analyzing the expression quantity of the target detection object; IV. comparing to find out whether the target detection object contains the above-mentioned biomarker; and V. comparing to find a symptom of Parkinson's disease corresponding to the biomarker.

In one aspect of the present invention, the specimen is blood or a brain tissue.

Another aspect of the present invention provides a kit comprising a reagent of the above-mentioned biomarkers.

The advantages of the present invention are as follows.
1. The biomarker of the present invention is different from the common markers in the past. It is a newly discovered marker that can effectively distinguish PD patients with different symptoms, so that the patients can receive more precise treatment as early as possible.
2. The biomarker of the present invention can bind with a clinical reagent to reduce the analysis time required for detection and meet the requirements for diagnosing Parkinson's disease.
3. Unifying the process of patient clinical diagnosis and plasma sampling methods facilities reducing operational variability and ensuring the consistency of sample storage, thereby ensuring the effective utilization of medical resources, and reducing the waste of medical resources.

### BRIEF DESCRIPTION OF THE DRAWINGS

The implementation aspects of the technology of the present invention will now be described by way of example only with reference to the accompanying drawings, in which:

FIG. 1 is a flow chart of screening of a piRNA biomarker.

FIGS. 2A to 2C are diagrams showing classification results of biomarkers according to an embodiment of the present invention.

It should be understood that aspects of the present invention are not limited to the configurations, means and characteristics shown in the accompanying drawings.

### DESCRIPTION OF THE EMBODIMENTS

All technical and scientific terms used herein have the same meaning as commonly understood by those of ordinary skills in the art to which the present invention belongs, unless otherwise defined. The following terms used throughout the present application shall have the following meanings.

“Or” represents “and/or”, unless otherwise stated. “Comprising” means not excluding the presence or addition of one or more other components, steps, operations or elements respectively to the described components, steps, operations or elements. The “comprising”, “including”, “containing”, “encompassing” and “having” described herein are interchangeable and not limiting. As used herein and in the appended claims, the singular forms “a”, “an” and “the” include plural referents, unless the context dictates otherwise. For example, the terms “a”, “an”, “the”, “one or more”, and “at least one” may be used interchangeably herein.

An aspect of the present invention provides a biomarker for differential diagnosis of a patient with Parkinson's disease, wherein the biomarker is piRNA, and is derived from plasma or from plasma and brain, or the biomarker is targeted to the brain. “Diagnosing Parkinson's disease” as used herein refers to determining whether an individual has Parkinson's disease, and also to determining the risk of an individual of developing Parkinson's disease.

piRNA is a small noncoding RNA that plays a crucial role in transcriptional gene silencing and post-transcriptional gene silencing, and that is involved in the regulation of gene expression and maintenance of genome integrity. The inventor of the present application has screened out piRNAs that can target Parkinson's disease as biomarkers through a series of analyses, thereby classifying PD patients with different symptoms and improving the accuracy of diagnosis of atypical Parkinson's disease. “Atypical Parkinson's disease” as used herein refers to Parkinson's disease with non-motor symptoms, such as PD with dementia (PDD), PD with no dementia (PDND), PD with no cognitive impairment (PDNC), or PD with mild cognitive impairment (PD-MCI).

The process of screening a biomarker related to PD of the present invention includes the following steps: sampling to detect hundreds of patients with Parkinson's disease, and diagnosing based on clinical diagnostic items such as MDS-UPDRS, MoCA, and MMSE, etc.; collecting information of the patients, such as: age, gender, education level, living habits, medical history, diet, exercise, patients are grouped according to different symptoms of Parkinson's disease; and then blood is drawn from the patients and the plasma is separated to analyze relevant biomarkers.

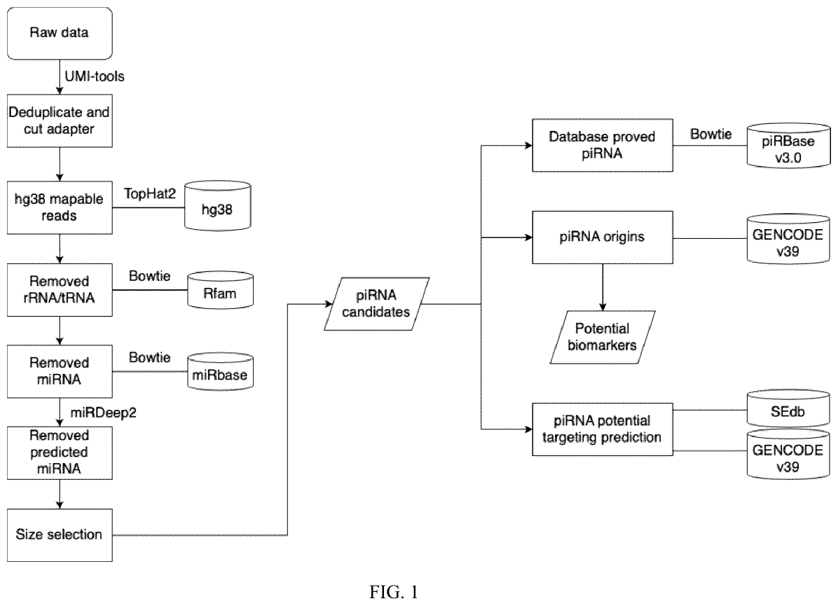

Please refer to FIG. 1. The analysis process of piRNA is as follows: after extracting RNA from peripheral blood plasma samples, and small RNA sequencing library is constructed with UMI barcodes to enable later deduplications to avoid disproportional amplification. The resulting small RNA sequencing result will be characterized, where a UMI tool is first utilized to remove duplications and cut adapters; then the processed data is subjected to comparison screening with human gene hg38 by Bowtie analysis; rRNA, tRNA and miRNA are removed from the screened data; and then small fragment RNA length screening is then performed to obtain piRNA candidates with 24 to 34 nucleotides. The obtained piRNA candidates are then analyzed by utilizing bioinformatics, and respectively compared with a piRNA database, screened for piRNA origins, and predicted for potential piRNAs targets; piRNA biomarkers are described using an entire host transcript derived piRNAs as a unit (using the gene name of the host transcript), or the group of piRNAs targeting the same RNA sequence as a unit (named after the predicted targeting transcript), or the group of piRNAs targeting specific enhancer, that are suitable for diagnosing Parkinson's disease are identified from the piRNA origins.

According to an embodiment of the present invention, the biomarker is derived from plasma, including all detectable piRNAs-like sequences categorized by their host transcript, including the following (Group 1): RNU2-51P, RNU6-727P, LDOC1, RNU2-16P, EYS, RNU6-37P, RNU2-60P, ENSG00000257657, U6, RNU6-3P, H3-3A, CCDC102B, RNU6-39P, RN7SL767P, FAM53A, PCDH11X, RNU2-26P, IFT88, RNU2-7P, INO80, KIF1B, RNU6-1225P, KTN1, HDAC1, UHRF1BP1, ANKHD1, ANKHD1-EIF4EBP3, RN7SL151P, ENSG00000285641, RAB13, RN7SL128P, VKORC1L1, EGR3, RNU1-2, RNY4P7, ENSG00000269707, POU3F3, WDR33, CROCC, Y_RNA, RN7SL87P, MEGF9, PGAP1, RNY4P25, PPHLN1, HS3ST4, SP3, RN7SL597P, RNU6-10P, TMOD3, GFM2, RNU6-658P, MAPT, FOXC1, CNTN3, SNORD57, CDK6, GPALPP1, RN7SL49P, AGBL5, H4C5, RN7SL4P, ZC3HAV1, RNU6-140P, EPB41, EIF3J, RUVBL1, RNU2-37P, RN7SL181P, ENSG00000288684, TOPORS, ULK4, ST6GAL2, ATP13A3, GRAP2, TRAK2, PIP4P1, RNU2-64P, ZNF385D, DUSP1, STAT5B, RGPD5, RNU6-743P, SNORD99, SNHG12, NBPF20, MANF, VTRNA1-1 or TBC1D14. In a preferred implementation aspect, the biomarker is RNU2-51P, RNU2-16P, POU3F3, WDR33, CROCC, PGAP1, MAPT or ATP13A3.

According to an embodiment of the present invention, the biomarkers of the aforementioned group (1) are used for distinguishing patients with non-Parkinson's disease (HC) from patients with Parkinson's disease (PD).

According to an embodiment of the present invention, the biomarker is derived from plasma and brain, including all detectable piRNAs-like sequences categorized by their host transcript, including the following (Group 2): LINC00910, RNU2-4P, CTSD, CCDC102B, MAPT, SEC14L1, PGAP1, RN7SL151P, PPHLN1, Y_RNA, C16orf72, ZC3HAV1, RNVU1-7, RN7SL767P, KLHL20, SGTA, DUSP3, KLF2, NUAK1, ACTR3, RAP1GDS1, ZFP36L2, PDXK, PTPRG, MN1, TMOD3, DPP10, TNRC6A, MARCHF8, CLIP4, CYBRD1, PPP3CA, POU3F2, FMNL3, GPATCH8, IQCH, USP7, RAB5C, STRBP, LARGE1, AKAP9, SHARPIN, NFIX, TOPORS, VKORC1L1, GPALPP1, LDB1, KIF21A, ST6GAL2, PARP1, ESRRG, PRRC2C, AFG1L, MBNL3, ERI3, MEGF9, SEMASA, RNU1-2, SNORD17, SPECC1, AKAP8L, ALKBH5, KIF2A, CHKB, GIGYF2, GNG12-AS1, CD99P1, COMMD3-BMI1, ANO6, ATP13A3, HLA-B, DENND4C, GNB1, TMA7, NCOR2, UBAC2, SHANK2, RPL13A, HS3ST4, NOTCH2, CHKB-CPT1B, SEC31A, TRIO, CSDE1, NAP1L1, HSP90B1, PPP1CC, RNF213, ZNF592, RN7SL49P, TMEM185A, MARCKS, LRPAP1, BCL2L1, MACF1, CWC27, BNIP3L, CATSPER2, FBXO7, HAUS7, HDAC1, HDAC8, KTN1, RN7SL1, RNU2-36P, SLC24A3, TPT1 or TREX2. In a preferred implementation aspect, the biomarker is LINC00910, PGAP1, MAPT or ATP13A3.

According to an embodiment of the present invention, the biomarkers of the aforementioned group (2) are used for distinguishing HC patients from PD patients, and the PD patients are patients with PD with no dementia (PDND), PD with mild cognitive impairment (PD-MCI) or PD with dementia (PDD); where the biomarker is LINC00910, RNU2-4P, CTSD, CCDC102B, MAPT, SEC14L1, PGAP1, RN7SL151P, PPHLN1, Y_RNA, C16orf72, ZC3HAV1, RNVU1-7, RN7SL767P, KLHL20, SGTA, DUSP3, KLF2, NUAK1, ACTR3, RAP1GDS1, ZFP36L2, PDXK, PTPRG, MN1, TMOD3, DPP10, TNRC6A, MARCHF8, CLIP4, CYBRD1, PPP3CA, POU3F2, FMNL3, GPATCH8, IQCH, USP7, RAB5C, STRBP, LARGE1, AKAP9, SHARPIN, NFIX, TOPORS, VKORC1L1, GPALPP1, LDB1, KIF21A, ST6GAL2, PARP1, ESRRG, PRRC2C, AFG1L, MBNL3, ERI3, MEGF9, SEMASA, RNU1-2, SNORD17, SPECC1, AKAP8L, ALKBH5, KIF2A, CHKB, GIGYF2, GNG12-AS1, CD99PI, COMMD3-BMI1, ANO6, ATP13A3, HLA-B, DENND4C, GNB1, TMA7, NCOR2, UBAC2, SHANK2, RPL13A, HS3ST4, NOTCH2, CHKB-CPT1B, SEC31A, TRIO, CSDE1, NAP1L1, HSP90B1, PPP1CC, RNF213, ZNF592, RN7SL49P, TMEM185A, MARCKS, LRPAP1, BCL2L1, MACF1 or CWC27.

In another embodiment aspect, the biomarkers of the aforementioned group (2) are used for distinguishing HC and PD patients, and the PD patients are PDND or PDD patients; wherein the biomarker is LINC00910, RNU2-4P, RN7SL1, RNU2-36P, HDAC8, TREX2, HAUS7, HDAC1, RN7SL151P, SLC24A3, ZC3HAV1, MAPT, FBXO7, TMOD3, CATSPER2, PGAP1, CCDC102B, TOPORS, VKORC1L1, GPALPP1, MEGF9, TPT1, RNVU1-7, RNU1-2, Y_RNA, KTN1, RN7SL767P, BNIP3L, RN7SL49P, TMEM185A, ATP13A3 or BCL2L1.

According to an embodiment of the present invention, the biomarker is derived from plasma, including all detectable piRNAs-like sequences categorized by their host transcript, including the following (Group 3): ZC3HAV1, CDK6, VKORC1L1, RAB13, MEGF9, RNU6-10P, RNU2-51P, U2, FYB1, UNC13C, THSD4, ZYX, CLCN3, PGAP1, SNX6, RAB13, GCNT1, Y_RNA, M6PR, VPS13D, TNFRSF1A, PAPOLA, EFS, AGO2, ATE1, AFM, IL10RA, ENO2, EXOC2, GCA, ITGB1BP1, ATAD2B, ACHE, INSIG2, GNG11, TUBGCP2, AHNAK, NT5E, GDF11, TTYH3, TANK, ATF7IP, CORO1C, LPCAT3, CERT1, ARL1, ATP5F1B, KCNH2, PIK3R3, LUC7L2, STAM2, AGPS, HERC5, PDCD6, ENPEP, RBM27, PIP5K1B, BLVRA, TCIRG1, SLC18A1, RBM6, ETNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIPI2, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, VWC2L, POC1B, H2BC4, GPHN, ANO1, CDHR3, ZFYVE1, PTPN6, ETS1, HTR1F, TAGLN2, ZBTB38, ZNF513, DTX2, CUL1, SCMH1, BTNL9, COA5, EEF1G, CABCOCO1, MRTO4, CD8B, SLC6A17, CEP63, WSCD2, TSKU, TESK1, DHX15, FGD3, ZNF385A, SLA, TMEM167B, CPM, FBXL14, RIMS2, ADIPOR1, ATP2B4, NHSL1, OAS2, CCDC18, NKX2-3, LPAR6, ATG16L1, SCEL, C7orf25, SPATA16, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, PIK3CG, LRP10, SLC39A10, RPL37A, TIAM2, UBAP2, NCKAP1, CLSTN1, RNUSB-1, RNU6-1309P, RYR2, LGALS8, DNAH14, TMEM181, MDM4, TOR1AIP2, RABGAP1L, IFNGR1, HBS1L, TMCO1, ARHGAP30, SELENBP1, PDSS2, PNISR, CD58, DUSP5, SUFU, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, API5, B3GALT6, SLC7A1, VGLL3, RNU6-629P, SNORD26, RNU6-43P, SNORD64, IGKV3-15, CCDC50, NUDT14, ARHGAP9, SYNE2, LBH, RASGEF1A, DDHD2, GNAT3, RPS6KA2, KMT5B, FAT3, MXD1, RNU2-29P, RNU2-59P, CROCCP2, NBEAL2, KBTBD8, NEBL, ISY1-RAB43, ACTG1P19, HTATIP2, THEM7P, ATF7, BSN-DT, TMEM132D, LINC-PINT, INO80D, LINC00993, MTCO1P51, SOX5, AHCYP2, IRAK4, LINC01807, LRRC23, ANXA2P2, FHIP1A, ABTB2, TRABD2B, USP46, TAX1BP1-AS1, TRGV9, COL3A1, POTEI, HRAT17, ARHGAP25, CFLAR, RNU7-1, SNORD121B, RNU7-19P, NDUFA5, CAP1, TRA2B, EIF3B, CTBP2, GNAI2, ADGRL2, TP53BP1, CYB5R4, FLNB, AOAH, ENPP7P4, UBE4B, RHOBTB1, RN7SL242P, LCP1, HNRNPA2BI, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, PABPC1, SLC45A4, CLU, SOX6, KMT2A, E2F8, XKR6, GALNT4, SF3B2, DLG2, GLYATL2, SNHG1, LINC02376, CUL3, C12orf57, FMC1-LUC7L2, POC1B-GALNT4, LINC02416, SNHG14, ACTN1, LINC00639, CCDC85C, MARK3, LINC02533, ATF7-NPFF, H4C1, WASF2, PWAR5, PLCL1, MAMDC2-AS1, SYNGAP1-AS1, RYR3, LINC00862, PHGDH, LINC02752, LINC01117, JAK1, PRKN, GSK3B, DDX54, LONRF2, Metazoa_SRP, PRDM10, RARB, CADM2, CMC1, EIF3A, FRMD4A, LINC01170, LINC02540, MIPOL1, NIBAN1, PPHLN1, PPTC7, RNU6-1062P, RNU6-84P, SNORD6, SORCS3, TAF1D, VWF or YY1. In a preferred embodiment aspect, the biomarker is RNU2-51P or PGAP1.

According to an embodiment of the present invention, the biomarkers of the aforementioned group (3) are used for distinguishing HC and PD patients, and the PD patients are PDND patients; wherein the biomarker is ZC3HAV1, CDK6, VKORC1L1, RAB13, MEGF9, RNU6-10P, RNU2-51P, U2 or FYB1. In another embodiment aspect, the biomarkers of the aforementioned group (3) are used for distinguishing HC and PD patients, and the PD patients are PD-MCI patients; wherein the biomarker is UNC13C, THSD4, ZYX, CLCN3, PGAP1, SNX6, RAB13, MEGF9, GCNT1 or Y_RNA. In another embodiment aspect, the biomarkers of the aforementioned group (3) are used for distinguishing HC and PD patients, and the PD patients are PDD patients; wherein the biomarker is M6PR, VPS13D, TNFRSF1A, PAPOLA, EFS, AGO2, ATE1, AFM, IL10RA, ENO2, EXOC2, GCA, ITGB1BP1, ATAD2B, ACHE, INSIG2, GNG11, TUBGCP2, AHNAK, NT5E, GDF11, TTYH3, TANK, ATF7IP, CORO1C, LPCAT3, CERT1, ARL1, ATP5F1B, KCNH2, PIK3R3, LUC7L2, STAM2, AGPS, HERC5, PDCD6, ENPEP, RBM27, PIP5K1B, BLVRA, TCIRG1, SLC18A1, RBM6, ETNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIPI2, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, VWC2L, POC1B, H2BC4, GPHN, ANO1, CDHR3, ZFYVE1, PTPN6, ETS1, HTR1F, TAGLN2, ZBTB38, ZNF513, DTX2, CUL1, SCMH1, BTNL9, COA5, EEF1G, CABCOCO1, MRTO4, CD8B, SLC6A17, CEP63, WSCD2, TSKU, TESK1, DHX15, FGD3, ZNF385A, SLA, TMEM167B, CPM, FBXL14, RIMS2, ADIPOR1, ATP2B4, NHSL1, OAS2, CCDC18, NKX2-3, LPAR6, ATG16L1, SCEL, C7orf25, SPATA16, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, PIK3CG, LRP10, SLC39A10, RPL37A, TIAM2, UBAP2, NCKAP1, CLSTN1, Y_RNA, RNUSB-1, RNU6-1309P, RYR2, LGALS8, DNAH14, TMEM181, MDM4, TOR1AIP2, RABGAP1L, IFNGR1, HBS1L, TMCO1, ARHGAP30, SELENBP1, PDSS2, PNISR, CD58, DUSP5, SUFU, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, API5, B3GALT6, SLC7A1, VGLL3, RNU6-629P, SNORD26, RNU6-43P, SNORD64, IGKV3-15, CCDC50, NUDT14, ARHGAP9, SYNE2, LBH, RASGEF1A, DDHD2, GNAT3, RPS6KA2, KMT5B, FAT3, MXD1, RNU2-29P, RNU2-59P, CROCCP2, NBEAL2, KBTBD8, NEBL, ISY1-RAB43, ACTG1P19, HTATIP2, THEM7P, ATF7, BSN-DT, TMEM132D, LINC-PINT, INO80D, LINC00993, MTCO1P51, SOX5, AHCYP2, IRAK4, LINC01807, LRRC23, ANXA2P2, FHIP1A, ABTB2, TRABD2B, USP46, TAX1BP1-AS1, TRGV9, COL3A1, POTEI, HRAT17, ARHGAP25, CFLAR, RNU7-1, SNORD121B, RNU7-19P, NDUFA5, CAP1, TRA2B, EIF3B, CTBP2, GNAI2, ADGRL2, TP53BP1, CYB5R4, FLNB, AOAH, ENPP7P4, UBE4B, RHOBTB1, RN7SL242P, LCP1, HNRNPA2BI, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, PABPC1, SLC45A4, CLU, SOX6, KMT2A, E2F8, XKR6, GALNT4, SF3B2, DLG2, GLYATL2, SNHG1, LINC02376, CUL3, C12orf57, FMC1-LUC7L2, POC1B-GALNT4, LINC02416, SNHG14, ACTN1, LINC00639, CCDC85C, MARK3, LINC02533, ATF7-NPFF, H4C1, WASF2, PWAR5, PLCL1, MAMDC2-AS1, SYNGAP1-AS1, RYR3, LINC00862, PHGDH, LINC02752, LINC01117, JAK1, PRKN or GSK3B.

According to an embodiment of the present invention, the biomarkers of the aforementioned group (3) are used for distinguishing PDND and PD-MCI patients, wherein the biomarker is UNC13C, PRDM10, DDX54, ZYX, CLCN3, SNX6, Y_RNA, GCNT1, RARB, LONRF2, RNU2-51P, SNHG14 or Metazoa_SRP. In another embodiment aspect, the biomarkers of the aforementioned group (3) are used for distinguishing PDND and PDD patients; wherein the biomarker is M6PR, VPS13D, TNFRSF1A, PAPOLA, EFS, AGO2, ATE1, AFM, IL10RA, ENO2, EXOC2, GCA, ITGB1BP1, ATAD2B, ACHE, INSIG2, GNG11, TUBGCP2, AHNAK, NT5E, GDF11, TTYH3, TANK, ATF7IP, CORO1C, VWF, LPCAT3, CERT1, ARL1, ATP5F1B, KCNH2, YY1, PIK3R3, STAM2, AGPS, PDCD6, ENPEP, RBM27, PIP5K1B, BLVRA, TCIRG1, SLC18A1, RBM6, ETNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIPI2, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, WC2L, POC1B, PPHLN1, CDHR3, PABPC1, ZFYVE1, PTPN6, ETS1, HTR1F, TAGLN2, ZBTB38, ZNF513, TAF1D, DTX2, CUL1, RABGAP1L, SCMH1, MIPOL1, BTNL9, COA5, EEF1G, CABCOCO1, MRTO4, CD8B, SLC6A17, CEP63, WSCD2, TSKU, CMC1, TESK1, DHX15, FGD3, ZNF385A, SLA, TMEM167B, CPM, FBXL14, RIMS2, ADIPOR1, ATP2B4, NHSL1, OAS2, CCDC18, NKX2-3, LPAR6, ATG16L1, SCEL, C7orf25, PPTC7, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, LRP10, SLC39A10, RPL37A, TIAM2, UBAP2, EIF3B, NCKAP1, CLSTN1, Y_RNA, RNUSB-1, RNU6-1309P, SNORD6, RYR2, LGALS8, DNAH14, PRKN, TMEM181, MDM4, TOR1AIP2, IFNGR1, HBS1L, TMCO1, ARHGAP30, SELENBP1, PDSS2, EIF3A, PNISR, CD58, DUSP5, SORCS3, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, API5, B3GALT6, SLC7A1, VGLL3, CADM2, RNU6-629P, SNORD26, SNORD64, RYR3, IGKV3-15, CCDC50, NUDT14, ARHGAP9, SYNE2, LBH, RASGEF1A, DDHD2, GNAT3, CLU, RPS6KA2, KMT5B, FAT3, LINC00993, MTCO1P51, AHCYP2, IRAK4, LINC01807, LRRC23, FHIP1A, ABTB2, TRABD2B, TAX1BP1-AS1, TRGV9, COL3A1, POTEI, HRAT17, LINC02540, ARHGAP25, CFLAR, SNORD121B, RNU7-19P, GPHN, NDUFA5, TRA2B, PIK3CG, CTBP2, GNAI2, ADGRL2, FRMD4A, TP53BP1, CYB5R4, FLNB, AOAH, ENPP7P4, UBE4B, RHOBTB1, RN7SL242P, LCP1, HNRNPA2B1, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, SLC45A4, SOX6, DLG2, KMT2A, E2F8, XKR6, GALNT4, GLYATL2, SNHG1, LINC02376, CUL3, SOX5, POC1B-GALNT4, LINC02416, SNHG14, ACTN1, LINC00639, CCDC85C, MARK3, LINC02533, ATF7-NPFF, RNU6-84P, H4C1, WASF2, PWAR5, PLCL1, MAMDC2-AS1, LINC01170, SYNGAP1-AS1, LINC00862, PHGDH, LINC02752, LINC01117 or JAK1. In another embodiment aspect, the biomarkers of the aforementioned group (3) are used for distinguishing PD-MCI and PDD patients; wherein the biomarker is M6PR, VPS13D, TNFRSF1A, PAPOLA, EFS, AGO2, ATE1, AFM, ENO2, EXOC2, GCA, ITGB1BP1, ATAD2B, ACHE, INSIG2, GNG11, TUBGCP2, NT5E, TTYH3, TANK, ATF7IP, CORO1C, LPCAT3, CERT1, ARL1, ATP5F1B, KCNH2, YY1, PIK3R3, LUC7L2, STAM2, AGPS, HERC5, PDCD6, ENPEP, RBM27, PIP5K1B, BLVRA, TCIRG1, SLC18A1, RBM6, TNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIPI2, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, VWC2L, H2BC4, ANO1, PHLN1, CDHR3, ZFYVE1, PTPN6, HTR1F, TAGLN2, ZBTB38, ZNF513, DTX2, CUL1, RABGAP1L, SCMH1, MIPOL1, BTNL9, COA5, EEF1G, CABCOCO1, MRTO4, CD8B, SLC6A17, CEP63, WSCD2, TSKU, TESK1, DHX15, FGD3, ZNF385A, TMEM167B, CPM, RIMS2, ADIPOR1, ATP2B4, HSL1, OAS2, CCDC18, NKX2-3, LPAR6, ATG16L1, SCEL, C7orf25, SPATA16, PPTC7, PRPF40A, ARNT, FAR1, CACNA2D1, KPNA5, PPP4R2, ELAVL4, RC3H2, NFIA, TNKSIBP1, ECHDC2, TXNDC15, MARCHF5, GULP1, PIK3CG, LRP10, SLC39A10, RPL37A, UBAP2, NCKAP1, CLSTN1, Y_RNA, RNU5B-1, RNU6-1309P, RYR2, LGALS8, DNAH14, TMEM181, MDM4, TOR1AIP2, IFNGR1, HBS1L, TMCO1, SELENBP1, PDSS2, EIF3A, PNISR, CD58, DUSP5, SORCS3, SUFU, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, AP15, B3GALT6, SLC7A1, VGLL3, CADM2, RNU6-1062P, RNU6-629P, SNORD26, RNU6-43P, SNORD64, RYR3, IGKV3-15, CCDC50, NUDT14, ARHGAP9, SYNE2, LBH, RASGEF1A, DDHD2, GNAT3, CLU, RPS6KA2, KMT5B, ARHGAP25, FAT3, MXD1, RNU2-29P, RNU2-59P, CROCCP2, NBEAL2, KBTBD8, ISY1-RAB43, ACTG1P19, HTATIP2, THEM7P, ATF7, BSN-DT, TMEM132D, LINC-PINT, INO80D, LINC00993, MTCO1P51, SOX5, AHCYP2, IRAK4, LINC01807, LRRC23, ANXA2P2, FHIP1A, ABTB2, TRABD2B, TAX1BP1-AS1, TRGV9, POTEI, CUL3, HRAT17, LINC02540, CFLAR, RNU7-1, SNORD121B, RNU7-19P, NDUFA5, CAP1, TRA2B, EIF3B, CTBP2, GNA12, ADGRL2, FRMD4A, TP53BP1, CYB5R4, AOAH, ENPP7P4, UBE4B, RHOBTB1, RN7SL242P, HNRNPA2BI, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, PABPC1, SLA, SLC45A4, SOX6, IL10RA, DLG2, KMT2A, E2F8, XKR6, GLYATL2, SNHG1, LINC02376, C12orf57, FMC1-LUC7L2, GPHN, LINC02416, SNHG14, ACTN1, LINC00639, CCDC85C, MARK3, LINC02533, ATF7-NPFF, RNU6-84P, H4C1, WASF2, PWAR5, PLCL1, MAMDC2-AS1, LINC01170, SYNGAP1-AS1, LINC00862, PHGDH, LINC02752, LINC01117 or PRKN.

According to an embodiment of the present invention, the brain-derived piRNA biomarker candidates targeting transcripts expressed in the brain, categorized by the targeted gene name as the following (Group 4): NRSN1, APPL2, TNFAIP8, ATP11A, LINC00847, CASP10, SNX16, MTA3, GRAPL, SMARCA2, LYPD3, ZNF252P-AS1, IRF2, TM9SF2, UNC79, LDC1P, GPR180, EFCAB5, PARP16, SH3D19, VPS37A, THAP8, IQSEC2, PTPRD, WDR7, INO80D, POLR1B, CCDC85C, CPE, STK24, DAAM2-AS1, CTSZ, DNAJC9, HSDL2-AS1, CBR4, IDI2-AS1, ACO2, ADNP-AS1, DIP2C-AS1, LINC00624, MAX, SRGAP2-AS1, NMT1, KLC4, KMT2E, LINC00506, ZNF565, ERAP1, TICAM2-AS1, PIGH, UFL1-AS1, VIM-AS1, KCNQ1OT1, TCHP, RB1, LIX1L-AS1, KCNMA1-AS3, PIGC, CYB5D2, FAM184B, FAR2, CRLF3, VCAN-AS1, STRADB, FTX, SKP2, RBFOX1, RPTOR, GNG12-AS1, CCDC85C, IDI2-AS1, NRSN1, TNFAIP8, KIF27, MEF2A, MT-ND3, NTAQ1, SMU1, SPINT2, PREPL, YJU2, MT-CO3 or THSD1. In a preferred embodiment aspect, the biomarker is MT-CO3, SH3D19, TM9SF2, LYPD3, LINC00847, PREPL, YJU2, GPR180, IRF2, IQSEC2, ATP11A, GRAPL, CASP10, UNC79, SMARCA2, ZNF252P-AS1 or MTA3. “Target” described herein refers to that a Parkinson's disease-related piRNA will act on the location of the claimed gene, so that in the patients with Parkinson's disease, the expression quantity of these genes will be reduced compared with those in patients with non-Parkinson's disease, and thus these genes can be used for distinguishing whether an individual is a patient with Parkinson's disease, or as a target for treatment of the patients.

According to an embodiment of the present invention, the biomarkers of the aforementioned group (4) are used for distinguishing HC and PD patients, and the PD patients are PDND patients; wherein the biomarker is NRSN1, APPL2, TNFAIP8, ATP11A, LINC00847, CASP10, SNX16, MTA3, GRAPL, SMARCA2, LYPD3, ZNF252P-AS1, IRF2, TM9SF2, UNC79, LDC1P, GPR180, EFCAB5, PARP16, SH3D19, VPS37A, THAP8, IQSEC2, PTPRD, WDR7, INO80D, POLR1B, CCDC85C, CPE, STK24, DAAM2-AS1, CTSZ, DNAJC9, HSDL2-AS1, CBR4, IDI2-AS1, ACO2, ADNP-AS1, DIP2C-AS1, LINC00624, MAX, SRGAP2-AS1, NMT1, KLC4, KMT2E, LINC00506, ZNF565, ERAP1, TICAM2-AS1, PIGH, UFL1-AS1, VIM-AS1, KCNQ1OT1, TCHP, RB1, LIX1L-AS1, KCNMA1-AS3, PIGC, CYB5D2, FAM184B, FAR2, CRLF3, VCAN-AS1, STRADB, FTX, SKP2, RBFOX1, RPTOR or GNG12-AS1. In another embodiment aspect, the biomarkers of the aforementioned group (4) are used for distinguishing HC and PD patients, and the PD patients are PDD patients; wherein the biomarker is NRSN1, SGMS1-AS1, IRF2, LINC00847, SMARCA2, WDR87BP, LYPD3, ATP11A, APPL2, TM9SF2, GPR180, PREPL, YJU2, TNFAIP8, MT-CO3, FAT3, MTCO3P12, SH3D19, MT-TH, IQSEC2, UNC79, CCDC85C, LBHDI, SLC5A2, ARG2, GPR156, AUNIP, FOXM1, IDI2-AS1, TMEM165 or CIQTNF7-AS1. In another embodiment aspect, the biomarkers of the aforementioned group (4) are used for distinguishing PDND and PDD patients; wherein the biomarker is NTAQ1, SPINT2, KIF27, THSD1, SMU1, MT-ND3 or MEF2A.

The biomarker screened out by the present invention is piRNA, and the piRNA is derived from the genes listed in the aforementioned groups (1) to (4). Further, the origin of the piRNA is derived from plasma or brain, or is brain-targeting. Specifically, the piRNA targets chromosome 1, 5, 7, 14, 15, 16, 17, 18 or 19 of the brain, and more specifically targets a typical enhancer region; and the typical enhancer described herein is a cis-acting element of DNA that acts on a promoter to enhance transcription. Specifically, the piRNA targets and acts on sites 173863970 to 173867965 on chromosome 1, sites 181240093 to 181245121 on chromosome 5, sites 148940554 to 148941891 on chromosome 7, sites 32200845 to 32204313 on chromosome 14, sites 44535775 to 44538187, 64459933 to 64462900, 67838570 to 67844704, and 69403194 to 69422417 on chromosome 15, sites 66548858 to 66553242 and 86506821 to 86513455 on chromosome 16, sites 40218274 to 40226290 and 43265728 to 43300986 on chromosome 17, sites 55584818 to 55590650 on chromosome 18, or sites 1017289 to 1030512 on chromosome 19, of the brain.

More preferably, the piRNA targets chromosome 2, 6, 9, 11, 13, 15, 16 or 17 of the brain, more specifically, targets a super enhancer region; and the super enhancer site described herein refers to a region containing multiple enhancers that can bind with multiple transcription factors to activate transcription of a gene that determines a cell differentiation outcome. The piRNA targets sites 216477408 to 216507672 on chromosome 2, sites 1600795 to 1637180 on chromosome 6, sites 125631123 to 125663808 on chromosome 9, sites 62838886 to 62/843,266 on chromosome 11, sites 99949209 to 99998706 on chromosome 13, sites 91850749 to 91867850 and 101469363 to 101492524 on chromosome 15, sites 86506821 to 86513455 on chromosome 16, or sites 7883182 to 7890259, 43231397 to 43252437, and 43265679 to 43315937 on chromosome 17, of the brain.

Another aspect of the present invention provides a method for detecting whether an individual has Parkinson's disease, including the following steps: I. acquiring a specimen from an individual; II. separating a target detection object from the specimen; III. analyzing the expression quantity of the target detection object; IV. comparing to find out whether the target detection object contains the biomarker as described above; and V. comparing to find a symptom of Parkinson's disease corresponding to the biomarker.

Specifically, the step I is to obtain a specimen from an individual, which specimen is body fluid, blood, saliva, urine, tears, semen, feces, a brain tissue, etc.; and in a preferred embodiment, the specimen is the blood or the brain tissue. Step II is to separate a target detection object from the specimen. The separation method can be using a TRIzol reagent, a mirVana miRNA Isolation Kit or a Qiagen miRNeasy Mini Kit; and in a preferred embodiment, it is to use a Qiagen miRNeasy Mini Kit. Step III is to analyze the expression quantity of the target detection object. The analysis method may be an analysis method well known to those skilled in the art, such as qPCR and next generation sequencing, etc. Steps IV and V are to compare to find whether the target detection object contains the biomarker as described above, and compare to find a symptom of Parkinson's disease corresponding to the biomarker, so as to determine the category of Parkinson's disease to which the individual belongs.

“Individual” as used herein refers to mammals, including but not limited to: human, non-human primates, sheep, dogs, murine rodents (e.g.: mice, rats, etc.), guinea pigs, cats, rabbits, cattle, horses. The aforementioned non-mammals include but are not limited to: chickens, amphibians and reptiles. Preferably, the individual is human.

According to an embodiment of the present invention, if the target detection object is piRNA, a method of analyzing the expression quantity of it is to conduct sequencing of piRNA by using a next-generation sequencing platform, e.g., pyrosequencing, with deoxynucleotides carrying 4 different bases being introduced sequentially during the process. When a nucleic acid polymerase joins the deoxynucleotides, pyrophosphates are released. The released pyrophosphates are converted by ATP sulfurylase to produce ATP, and then the energy of the ATP is received by luciferase to oxidize luciferin, and finally a signal is measured by a sensor to obtain a sequencing result; alternatively, small nucleic acid fragments that have been connected to an adapter are injected onto a chip of which the surface carries a complementary sequence of the adapter, so that each small nucleic acid fragment seems to be seeded on the chip, and then bridge amplification is performed on the solid surface of the chip, reagents containing 4 kinds of removable deoxyribonucleotides (dNTPs) with different fluorescence labels for PCR reaction. Once dNTP is connected to a DNA fragment being synthesized, a fluorescent molecule will be released and then the image is detected. The fluorescent labelling and removing, reagent replacement and image detection will be repeated to obtain a sequencing result. Chylous nucleic acid template amplification technology (emulsion PCR), firstly amplifies nucleic acid fragments on Ion Sphere Particles, and then puts the Ion Sphere Particles into a semiconductor wafer for sequencing; alternatively, 4 dNTPs are respectively injected during sequencing, hydrogen ions will be released when joining is conducted by the polymerase, resulting in a change in potential difference, so that a sensing element in the semiconductor wafer can judge which bases are A, T, C and G.

According to an embodiment of the present invention, if the specimen is blood, the time for allowing the plasma sample to stand can be within 1 to 3 hours, for example: within 1 hour, within 2 hours and within 3 hours. In a preferred embodiment aspect, the plasma sample is allowed to stand for 3 hours.

According to an embodiment of the present invention, the symptoms of Parkinson's disease include Parkinson's disease (PD), PD with dementia (PDD), PD with no dementia (PDND), PD with no cognitive impairment (PDNC) or PD with mild cognitive impairment (PD-MCI).

According to an embodiment of the present invention, the present invention further provides a therapeutic target, wherein the therapeutic target is selected from the biomarkers of group (4).

A further aspect of the present invention provides a kit including a reagent for the biomarker as described above. The kit can be used for diagnosing whether the user has Parkinson's disease, and can also distinguish Parkinson's disease with different symptoms, which can meet the requirements for early diagnosis of Parkinson's disease, thereby reducing the medical burden on society.

### EXAMPLES

It should be understood that the examples and embodiments described herein are for illustrative purposes only, and that various modifications or changes to the examples and embodiments will be suggested to those skilled in the art, and that such modifications or changes will be included within the spirit and scope of the present application and the scope of the appended claims. All publications, patents, and patent applications cited herein are hereby incorporated by reference in their entirety for all purposes.

### Example 1. Screening piRNA as a Biomarker

This experiment provided a process for screening a piRNA as a biomarker for detecting whether an individual has Parkinson's disease, which specifically included the following steps:

Firstly, 61 subjects were recruited and diagnosed according to clinical diagnostic items such as MDS-UPDRS, MoCA, and MMSE, etc. The patients were grouped according to different symptoms of Parkinson's disease. Blood was drawn from the subjects and respectively added into vacuum blood collection tubes containing EDTA, and allowed to stand for 3 hours. A total of 61 tubes of plasma samples were collected.

Subsequently, the small RNAs in the 61 tubes of plasma samples were separated from plasma by a Qiagen miRNeasy kit, and the small RNAs were sequenced by Illumina MiSeq. Subsequently, the bioinformatics process as shown in FIG. 1 was utilized to isolate piRNA candidates, and the piRNA-derived transcripts were calculated by piRPM (piRNA readings per million readings). Statistical analysis was performed to generate biomarkers derived from the piRNA transcripts.

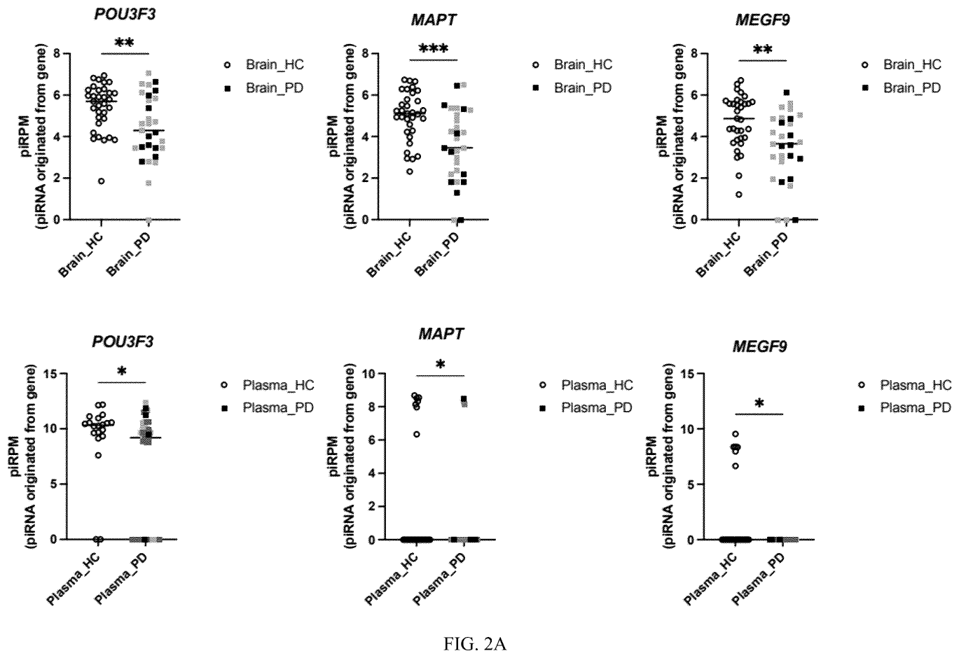

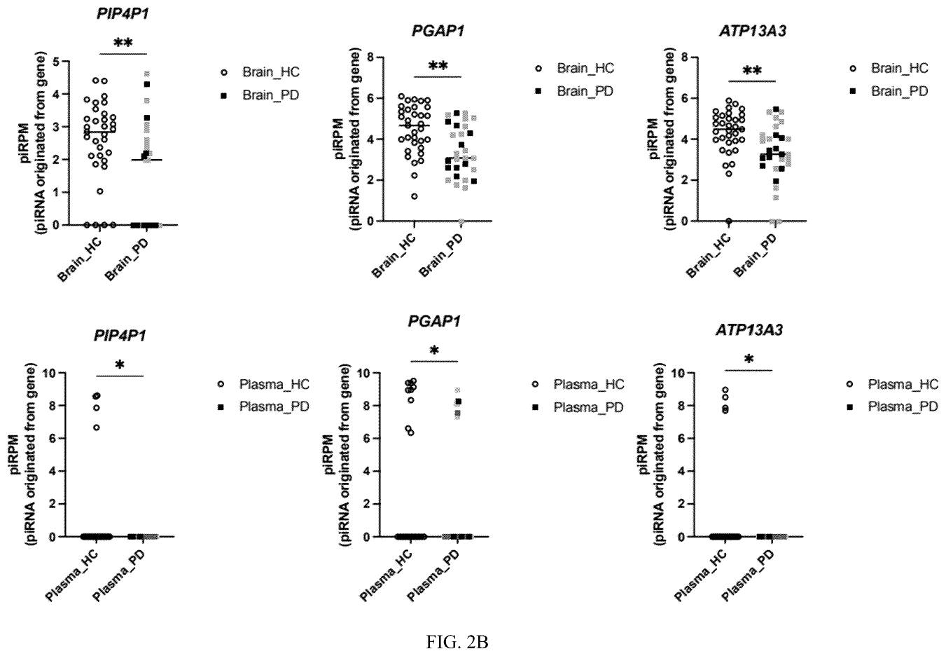

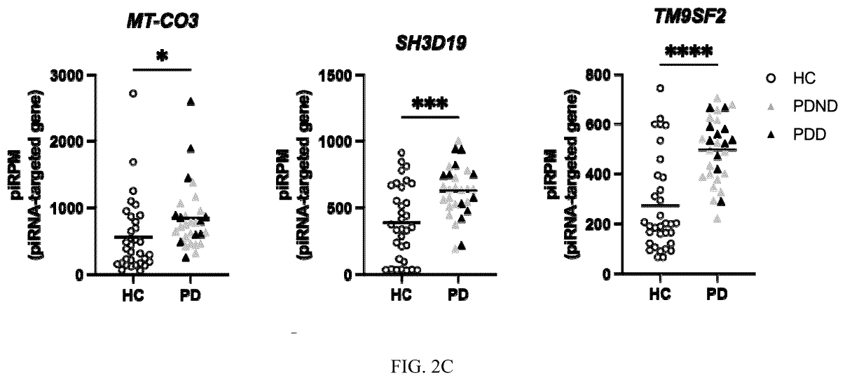

Finally, a piRNA isolated from the 61 tubes of plasma samples was sequenced by the next generation gene sequencing platform, and the expression quantity thereof was analyzed. By Anova analysis, when a p value was significantly different, the piRNA was used as a biomarker. This experiment also used data from an NCBI public platform, data of 62 small fragments of brain tissues derived from healthy people and patients with Parkinson's disease, combined with the plasma samples of the present invention for analysis, and to find out a target gene location that could serve as a biomarker and a treatment guide. Results were as shown in FIGS. 2A to 2B. These biomarkers could be used for differential diagnosis, effectively distinguishing patients with non-Parkinson's disease (HC) from patients with Parkinson's disease (PD). FIG. 2C and Table 1 showed that these biomarkers were predicted potential piRNA targets and could be used as therapeutic targets; wherein the patients with Parkinson's disease (PD) could be in turn divided into PDND or PDD with different symptoms. Specifically, these piRNAs targeted the locations of the genes listed in FIG. 2C and Table 1. Therefore, in the patients with Parkinson's disease, the expression quantities of these genes were reduced compared to those in healthy subjects, and thus they could be used as a biomarker or a targeted target for treating a patient.

TABLE 1

| | HC | PDND | PDD | p value |
|---|---|---|---|---|
| LYPD3 | 37.53 ± 33.09 | 60.17 ± 26.15 | 73.23 ± 35.37 | 0.0011 |
| LINC00847 | 18.51 ± 12.60 | 57.21 ± 31.55 | 40.32 ± 26.04 | <0.0001 |
| PREPL | 31.18 ± 35.18 | 45.45 ± 13.82 | 55.67 ± 27.51 | 0.0147 |
| YJU2 | 28.55 ± 27.33 | 42.39 ± 17.35 | 55.90 ± 24.16 | 0.0030 |
| GPR180 | 19.73 ± 18.01 | 34.65 ± 16.68 | 42.75 ± 16.07 | 0.0001 |
| IRF2 | 18.14 ± 16.99 | 29.49 ± 15.44 | 33.93 ± 10.82 | 0.0015 |
| IQSEC2 | 16.33 ± 13.65 | 27.27 ± 12.47 | 27.31 ± 7.63 | 0.0008 |
| ATP11A | 9.03 ± 7.82 | 37.08 ± 30.44 | 29.10 ± 19.69 | <0.0001 |
| GRAPL | 10.66 ± 6.85 | 27.81 ± 19.10 | 17.63 ± 13.14 | 0.0005 |
| CASP10 | 7.61 ± 7.35 | 21.36 ± 14.47 | 15.45 ± 9.48 | 0.0001 |
| UNC79 | 8.74 ± 6.41 | 17.26 ± 7.31 | 14.58 ± 8.02 | 0.0001 |
| SMARCA2 | 7.67 ± 6.31 | 21.51 ± 15.02 | 18.11 ± 12.60 | <0.0001 |
| ZNF252P- | 6.79 ± 5.20 | 22.20 ± 15.73 | 14.71 ± 10.94 | <0.0001 |
| AS1 | | | | |
| MTA3 | 7.23 ± 5.62 | 18.38 ± 13.14 | 11.81 ± 10.36 | 0.0014 |

### Example 2: Utilizing the Aforementioned piRNA to Detect Whether an Individual has Parkinson's Disease and Utilizing piRNA Targeting as a Therapeutic Target

piRNAs in plasma of an individual was analyzed by utilizing the aforementioned steps, and further comparison was conducted to find out whether the target detection object contained the biomarker of the present invention. A background noise value of the target detection object in the plasma sample was adjusted by the Anova analysis method, so as to highlight a biomarker in these plasma samples that can be used for differential diagnosis of a patient with Parkinson's disease; wherein the biomarker that could be used for differential diagnosis of a patient with Parkinson's disease is selected from group (1), (2), (3) or (4). Finally, the biomarker in the target detection object was compared with those in groups (1) to (4), and further comparison was conducted to find out the biomarker was used for distinguishing which type of Parkinson's disease, so as to obtain the category of the Parkinson's disease in the individual. Further, the biomarkers in group (4) could also be used as targets for treating Parkinson's disease. For example, treatment against piRNAs targeting LYPD3 may be conducted to prevent Parkinson's disease-related piRNA from targeting and degrading LYPD3, thereby reducing the expression quantity of LYPD3.

To sum up, by using the biomarker of the present invention, the patients with Parkinson's disease can be classified in advance, so as to improve the treatment efficiency and improve the demand for differential diagnosis of patients with atypical Parkinson's disease in the current medical field.

The present invention has been described in detail above. However, the above description is only the preferred embodiments of the present invention. They should not be considered as limiting the scope of the present invention. That is, all equivalent changes and modifications made according to the claims of the present invention should still fall within the patent coverage scope of the present invention.

What is claimed is:

1. A biomarker for differential diagnosis of a patient with Parkinson's disease, wherein the biomarker is piRNA, and is derived from plasma or from plasma and brain, or the biomarker is targeting brain related genes.

2. The biomarker according to claim 1, wherein the biomarker is derived from plasma, including all detectable piRNAs-like sequences categorized by their host transcript, including the following (Group 1): RNU2-51P, RNU6-727P, LDOC1, RNU2-16P, EYS, RNU6-37P, RNU2-60P, ENSG00000257657, U6, RNU6-3P, H3-3A, CCDC102B, RNU6-39P, RN7SL767P, FAM53A, PCDH11X, RNU2-26P, IFT88, RNU2-7P, INO80, KIF1B, RNU6-1225P, KTN1, HDAC1, UHRF1BP1, ANKHD1, ANKHD1-EIF4EBP3, RN7SL151P, ENSG00000285641, RAB13, RN7SL128P, VKORC1L1, EGR3, RNU1-2, RNY4P7, ENSG00000269707, POU3F3, WDR33, CROCC, Y_RNA, RN7SL87P, MEGF9, PGAP1, RNY4P25, PPHLN1, HS3ST4, SP3, RN7SL597P, RNU6-10P, TMOD3, GFM2, RNU6-658P, MAPT, FOXC1, CNTN3, SNORD57, CDK6, GPALPP1, RN7SL49P, AGBL5, H4C5, RN7SL4P, ZC3HAV1, RNU6-140P, EPB41, EIF3J, RUVBL1, RNU2-37P, RN7SL181P, ENSG00000288684, TOPORS, ULK4, ST6GAL2, ATP13A3, GRAP2, TRAK2, PIP4PI, RNU2-64P, ZNF385D, DUSP1, STAT5B, RGPD5, RNU6-743P, SNORD99, SNHG12, NBPF20, MANF, VTRNA1-1 or TBC1D14.

3. The biomarker according to claim 1, wherein the biomarker is derived from plasma and brain, including all detectable piRNAs-like sequences categorized by their host transcript, including the following (Group 2): LINC00910, RNU2-4P, CTSD, CCDC102B, MAPT, SEC14L1, PGAP1, RN7SL151P, PPHLN1, Y_RNA, C16orf72, ZC3HAV1, RNVU1-7, RN7SL767P, KLHL20, SGTA, DUSP3, KLF2, NUAK1, ACTR3, RAP1GDS1, ZFP36L2, PDXK, PTPRG, MN1, TMOD3, DPP10, TNRC6A, MARCHF8, CLIP4, CYBRD1, PPP3CA, POU3F2, FMNL3, GPATCH8, IQCH, USP7, RAB5C, STRBP, LARGE1, AKAP9, SHARPIN, NFIX, TOPORS, VKORC1L1, GPALPP1, LDB1, KIF21A, ST6GAL2, PARP1, ESRRG, PRRC2C, AFG1L, MBNL3, ERI3, MEGF9, SEMASA, RNU1-2, SNORD17, SPECC1, AKAP8L, ALKBH5, KIF2A, CHKB, GIGYF2, GNG12-AS1, CD99PI, COMMD3-BMI1, ANO6, ATP13A3, HLA-B, DENND4C, GNB1, TMA7, NCOR2, UBAC2, SHANK2, RPL13A, HS3ST4, NOTCH2, CHKB-CPT1B, SEC31A, TRIO, CSDE1, NAP1L1, HSP90B1, PPP1CC, RNF213, ZNF592, RN7SL49P, TMEM185A, MARCKS, LRPAP1, BCL2L1, MACF1, CWC27, BNIP3L, CATSPER2, FBXO7, HAUS7, HDAC1, HDAC8, KTN1, RN7SL1, RNU2-36P, SLC24A3, TPT1 or TREX2.

4. The biomarker according to claim 1, wherein the biomarker is derived from plasma, including all detectable piRNAs-like sequences categorized by their host transcript, including the following (Group 3): ZC3HAV1, CDK6, VKORC1L1, RAB13, MEGF9, RNU6-10P, RNU2-51P, U2, FYB1, UNC13C, THSD4, ZYX, CLCN3, PGAP1, SNX6, RAB13, GCNT1, Y_RNA, M6PR, VPS13D, TNFRSF1A, PAPOLA, EFS, AGO2, ATE1, AFM, IL10RA, ENO2, EXOC2, GCA, ITGB1BP1, ATAD2B, ACHE, INSIG2, GNG11, TUBGCP2, AHNAK, NT5E, GDF11, TTYH3, TANK, ATF7IP, CORO1C, LPCAT3, CERT1, ARL1, ATP5F1B, KCNH2, PIK3R3, LUC7L2, STAM2, AGPS, HERC5, PDCD6, ENPEP, RBM27, PIP5K1B, BLVRA, TCIRG1, SLC18A1, RBM6, ETNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIP12, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, VWC2L, POC1B, H2BC4, GPHN, ANO1, CDHR3, ZFYVE1, PTPN6, ETS1, HTR1F, TAGLN2, ZBTB38, ZNF513, DTX2, CUL1, SCMH1, BTNL9, COA5, EEF1G, CABCOCO1, MRTO4, CD8B, SLC6A17, CEP63, WSCD2, TSKU, TESK1, DHX15, FGD3, ZNF385A, SLA, TMEM167B, CPM, FBXL14, RIMS2, ADIPOR1, ATP2B4, NHSL1, OAS2, CCDC18, NKX2-3, LPAR6, ATG16L1, SCEL, C7orf25, SPATA16, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, PIK3CG, LRP10, SLC39A10, RPL37A, TIAM2, UBAP2, NCKAP1, CLSTN1, RNU5B-1, RNU6-1309P, RYR2, LGALS8, DNAH14, TMEM181, MDM4, TOR1AIP2, RABGAP1L, IFNGR1, HBS1L, TMCO1, ARHGAP30, SELENBP1, PDSS2, PNISR, CD58, DUSP5, SUFU, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, API5, B3GALT6, SLC7A1, VGLL3, RNU6-629P, SNORD26, RNU6-43P, SNORD64, IGKV3-15, CCDC50, NUDT14, ARHGAP9, SYNE2, LBH, RASGEF1A, DDHD2, GNAT3, RPS6KA2, KMT5B, FAT3, MXD1, RNU2-29P, RNU2-59P, CROCCP2, NBEAL2, KBTBD8, NEBL, ISY1-RAB43, ACTG1P19, HTATIP2, THEM7P, ATF7, BSN-DT, TMEM132D, LINC-PINT, INO80D, LINC00993, MTCO1P51, SOX5, AHCYP2, IRAK4, LINC01807, LRRC23, ANXA2P2, FHIP1A, ABTB2, TRABD2B, USP46, TAX1BP1-AS1, TRGV9, COL3A1, POTEI, HRAT17, ARHGAP25, CFLAR, RNU7-1, SNORD121B, RNU7-19P, NDUFA5, CAP1, TRA2B, EIF3B, CTBP2, GNA12, ADGRL2, TP53BP1, CYB5R4, FLNB, AOAH, ENPP7P4, UBE4B, RHOBTB1, RN7SL242P, LCP1, HNRNPA2B1, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, PABPC1, SLC45A4, CLU, SOX6, KMT2A, E2F8, XKR6, GALNT4, SF3B2, DLG2, GLYATL2, SNHG1, LINC02376, CUL3, C12orf57, FMC1-LUC7L2, POC1B-GALNT4, LINC02416, SNHG14, ACTN1, LINC00639, CCDC85C, MARK3, LINC02533, ATF7-NPFF, H4C1, WASF2, PWAR5, PLCL1, MAMDC2-AS1, SYNGAP1-AS1, RYR3, LINC00862, PHGDH, LINC02752, LINC01117, JAK1, PRKN, GSK3B, DDX54, LONRF2, Metazoa_SRP, PRDM10, RARB, CADM2, CMC1, EIF3A, FRMD4A, LINC01170, LINC02540, MIPOL1, NIBAN1, PPHLN1, PPTC7, RNU6-1062P, RNU6-84P, SNORD6, SORCS3, TAF1D, VWF or YY1.

5. The biomarker according to claim 1, wherein the brain-derived piRNA biomarker candidates targeting transcripts expressed in the brain, categorized by the targeted gene name as the following (Group 4): NRSN1, APPL2, TNFAIP8, ATP11A, LINC00847, CASP10, SNX16, MTA3, GRAPL, SMARCA2, LYPD3, ZNF252P-AS1, IRF2, TM9SF2, UNC79, LDC1P, GPR180, EFCAB5, PARP16, SH3D19, VPS37A, THAP8, IQSEC2, PTPRD, WDR7, INO80D, POLR1B, CCDC85C, CPE, STK24, DAAM2-AS1, CTSZ, DNAJC9, HSDL2-AS1, CBR4, IDI2-AS1, ACO2, ADNP-AS1, DIP2C-AS1, LINC00624, MAX, SRGAP2-AS1, NMT1, KLC4, KMT2E, LINC00506, ZNF565, ERAP1, TICAM2-AS1, PIGH, UFL1-AS1, VIM-AS1, KCNQ1OT1, TCHP, RB1, LIX1L-AS1, KCNMA1-AS3, PIGC, CYB5D2, FAM184B, FAR2, CRLF3, VCAN-AS1, STRADB, FTX, SKP2, RBFOX1, RPTOR, GNG12-AS1, CCDC85C, IDI2-AS1, NRSN1, TNFAIP8, KIF27, MEF2A, MT-ND3, NTAQ1, SMU1, SPINT2, PREPL, YJU2, MT-CO3 or THSD1.

6. The biomarker according to claim 2, wherein the biomarker is used for distinguishing patients with non-Parkinson's disease (HC) from patients with Parkinson's disease (PD).

7. The biomarker according to claim 2, wherein the biomarker is RNU2-51P, RNU2-16P, POU3F3, WDR33, CROCC, PGAP1, MAPT or ATP13A3.

8. The biomarker according to claim 3, wherein the biomarker is used for distinguishing HC patients from PD patients, and the PD patients are patients with PD with no dementia (PDND), PD with mild cognitive impairment (PD-MCI) or PD with dementia (PDD); where the biomarker is LINC00910, RNU2-4P, CTSD, CCDC102B, MAPT, SEC14L1, PGAP1, RN7SL151P, PPHLN1, Y_RNA, C16orf72, ZC3HAV1, RNVU1-7, RN7SL767P, KLHL20, SGTA, DUSP3, KLF2, NUAK1, ACTR3, RAP1GDS1, ZFP36L2, PDXK, PTPRG, MN1, TMOD3, DPP10, TNRC6A, MARCHF8, CLIP4, CYBRD1, PPP3CA, POU3F2, FMNL3, GPATCH8, IQCH, USP7, RAB5C, STRBP, LARGE1, AKAP9, SHARPIN, NFIX, TOPORS, VKORC1L1, GPALPP1, LDB1, KIF21A, ST6GAL2, PARP1, ESRRG, PRRC2C, AFG1L, MBNL3, ERI3, MEGF9, SEMASA, RNU1-2, SNORD17, SPECC1, AKAP8L, ALKBH5, KIF2A, CHKB, GIGYF2, GNG12-AS1, CD99P1, COMMD3-BMI1, ANO6, ATP13A3, HLA-B, DENND4C, GNB1, TMA7, NCOR2, UBAC2, SHANK2, RPL13A, HS3ST4, NOTCH2, CHKB-CPT1B, SEC31A, TRIO, CSDE1, NAP1L1, HSP90B1, PPP1CC, RNF213, ZNF592, RN7SL49P, TMEM185A, MARCKS, LRPAP1, BCL2L1, MACF1 or CWC27.

9. The biomarker according to claim 3, wherein the biomarker is used for distinguishing HC and PD patients, and the PD patients are PDND or PDD patients; wherein the biomarker is LINC00910, RNU2-4P, RN7SL1, RNU2-36P, HDAC8, TREX2, HAUS7, HDAC1, RN7SL151P, SLC24A3, ZC3HAV1, MAPT, FBX07, TMOD3, CATSPER2, PGAP1, CCDC102B, TOPORS, VKORC1L1, GPALPP1, MEGF9, TPT1, RNVU1-7, RNU1-2, Y_RNA, KTN1, RN7SL767P, BNIP3L, RN7SL49P, TMEM185A, ATP13A3 or BCL2L1.

10. The biomarker according to claim 3, wherein the biomarker is LINC00910, PGAP1, MAPT or ATP13A3.

11. The biomarker according to claim 4, wherein the biomarker is used for distinguishing HC and PD patients, and the PD patients are PDND patients; wherein the biomarker is ZC3HAV1, CDK6, VKORC1L1, RAB13, MEGF9, RNU6-10P, RNU2-51P, U2 or FYB1.

12. The biomarker according to claim 4, wherein the biomarker is used for distinguishing HC and PD patients, and the PD patients are PD-MCI patients; wherein the biomarker is UNC13C, THSD4, ZYX, CLCN3, PGAP1, SNX6, RAB13, MEGF9, GCNT1 or Y_RNA.

13. The biomarker according to claim 4, wherein the biomarker is used for distinguishing HC and PD patients, and the PD patients are PDD patients; wherein the biomarker is M6PR, VPS13D, TNFRSF1A, PAPOLA, EFS, AGO2, ATE1, AFM, IL10RA, ENO2, EXOC2, GCA, ITGB1BP1, ATAD2B, ACHE, INSIG2, GNG11, TUBGCP2, AHNAK, NT5E, GDF11, TTYH3, TANK, ATF7IP, CORO1C, LPCAT3, CERT1, ARL1, ATP5F1B, KCNH2, PIK3R3, LUC7L2, STAM2, AGPS, HERC5, PDCD6, ENPEP, RBM27, PIP5K1B, BLVRA, TCIRG1, SLC18A1, RBM6, ETNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIP12, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, VWC2L, POC1B, H2BC4, GPHN, ANO1, CDHR3, ZFYVE1, PTPN6, ETS1, HTR1F, TAGLN2, ZBTB38, ZNF513, DTX2, CUL1, SCMH1, BTNL9, COA5, EEF1G, CABCOCO1, MRTO4, CD8B, SLC6A17, CEP63, WSCD2, TSKU, TESK1, DHX15, FGD3, ZNF385A, SLA, TMEM167B, CPM, FBXL14, RIMS2, ADIPOR1, ATP2B4, NHSL1, OAS2, CCDC18, NKX2-3, LPAR6, ATG16L1, SCEL, C7orf25, SPATA16, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, PIK3CG, LRP10, SLC39A10, RPL37A, TIAM2, UBAP2, NCKAP1, CLSTN1, Y_RNA, RNU5B-1, RNU6-1309P, RYR2, LGALS8, DNAH14, TMEM181, MDM4, TOR1AIP2, RABGAP1L, IFNGR1, HBS1L, TMCO1, ARHGAP30, SELENBP1, PDSS2, PNISR, CD58, DUSP5, SUFU, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, API5, B3GALT6, SLC7A1, VGLL3, RNU6-629P, SNORD26, RNU6-43P, SNORD64, IGKV3-15, CCDC50, NUDT14, ARHGAP9, SYNE2, LBH, RASGEF1A, DDHD2, GNAT3, RPS6KA2, KMT5B, FAT3, MXD1, RNU2-29P, RNU2-59P, CROCCP2, NBEAL2, KBTBD8, NEBL, ISY1-RAB43, ACTG1P19, HTATIP2, THEM7P, ATF7, BSN-DT, TMEM132D, LINC-PINT, INO80D, LINC00993, MTCO1P51, SOX5, AHCYP2, IRAK4, LINC01807, LRRC23, ANXA2P2, FHIP1A, ABTB2, TRABD2B, USP46, TAX1BP1-AS1, TRGV9, COL3A1, POTEI, HRAT17, ARHGAP25, CFLAR, RNU7-1, SNORD121B, RNU7-19P, NDUFA5, CAP1, TRA2B, EIF3B, CTBP2, GNAI2, ADGRL2, TP53BP1, CYB5R4, FLNB, AOAH, ENPP7P4, UBE4B, RHOBTB1, RN7SL242P, LCP1, HNRNPA2B1, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, PABPC1, SLC45A4, CLU, SOX6, KMT2A, E2F8, XKR6, GALNT4, SF3B2, DLG2, GLYATL2, SNHG1, LINC02376, CUL3, C12orf57, FMC1-LUC7L2, POC1B-GALNT4, LINC02416, SNHG14, ACTN1, LINC00639, CCDC85C, MARK3, LINC02533, ATF7-NPFF, H4C1, WASF2, PWAR5, PLCL1, MAMDC2-AS1, SYNGAP1-AS1, RYR3, LINC00862, PHGDH, LINC02752, LINC01117, JAK1, PRKN or GSK3B.

14. The biomarker according to claim 4, wherein the biomarker is used for distinguishing PDND and PD-MCI patients, wherein the biomarker is UNC13C, PRDM10, DDX54, ZYX, CLCN3, SNX6, Y_RNA, GCNT1, RARB, LONRF2, RNU2-51P, SNHG14 or Metazoa_SRP.

15. The biomarker according to claim 4, wherein the biomarker is used for distinguishing PDND and PDD patients; wherein the biomarker is M6PR, VPS13D, TNFRSF1A, PAPOLA, EFS, AGO2, ATE1, AFM, IL10RA, ENO2, EXOC2, GCA, ITGB1BP1, ATAD2B, ACHE, INSIG2, GNG11, TUBGCP2, AHNAK, NT5E, GDF11, TTYH3, TANK, ATF7IP, CORO1C, VWF, LPCAT3, CERT1, ARL1, ATP5F1B, KCNH2, YY1, PIK3R3, STAM2, AGPS, PDCD6, ENPEP, RBM27, PIP5K1B, BLVRA, TCIRG1, SLC18A1, RBM6, ETNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIPI2, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, WC2L, POC1B, PPHLN1, CDHR3, PABPC1, ZFYVE1, PTPN6, ETS1, HTR1F, TAGLN2, ZBTB38, ZNF513, TAF1D, DTX2, CUL1, RABGAP1L, SCMH1, MIPOL1, BTNL9, COA5, EEF1G, CABCOCO1, MRTO4, CD8B, SLC6A17, CEP63, WSCD2, TSKU, CMC1, TESK1, DHX15, FGD3, ZNF385A, SLA, TMEM167B, CPM, FBXL14, RIMS2, ADIPOR1, ATP2B4, NHSL1, OAS2, CCDC18, NKX2-3, LPAR6, ATG16L1, SCEL, C7orf25, PPTC7, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, LRP10, SLC39A10, RPL37A, TIAM2, UBAP2, EIF3B, NCKAP1, CLSTN1, Y_RNA, RNU5B-1, RNU6-1309P, SNORD6, RYR2, LGALS8, DNAH14, PRKN, TMEM181, MDM4, TOR1AIP2, IFNGR1, HBS1L, TMCO1, ARHGAP30, SELENBP1, PDSS2, EIF3A, PNISR, CD58, DUSP5, SORCS3, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, AP15, B3GALT6, SLC7A1, VGLL3, CADM2, RNU6-629P, SNORD26, SNORD64, RYR3, IGKV3-15, CCDC50, NUDT14, ARHGAP9, SYNE2, LBH, RASGEF1A, DDHD2, GNAT3, CLU, RPS6KA2, KMT5B, FAT3, MXD1, RNU2-29P, CROCCP2, NBEAL2, NIBAN1, KBTBD8, NEBL, ISY1-RAB43, ACTG1P19, HTATIP2, THEM7P, ATF7, BSN-DT, TMEM132D, LINC-PINT, INO80D, LINC00993, MTCO1P51, AHCYP2, IRAK4, LINC01807, LRRC23, FHIP1A, ABTB2, TRABD2B, TAX1BP1-AS1, TRGV9, COL3A1, POTEI, HRAT17, LINC02540, ARHGAP25, CFLAR, SNORD121B, RNU7-19P, GPHN, NDUFA5, TRA2B, PIK3CG, CTBP2, GNAI2, ADGRL2, FRMD4A, TP53BP1, CYB5R4, FLNB, AOAH, ENPP7P4, UBE4B, RHOBTB1, RN7SL242P, LCP1, HNRNPA2BI, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, SLC45A4, SOX6, DLG2, KMT2A, E2F8, XKR6, GALNT4, GLYATL2, SNHG1, LINC02376, CUL3, SOX5, POC1B-GALNT4, LINC02416, SNHG14, ACTN1, LINC00639, CCDC85C, MARK3, LINC02533, ATF7-NPFF, RNU6-84P, H4C1, WASF2, PWAR5, PLCL1, MAMDC2-AS1, LINC01170, SYNGAP1-AS1, LINC00862, PHGDH, LINC02752, LINC01117 or JAK1.

16. The biomarker according to claim 4, wherein the biomarker is used for distinguishing PD-MCI and PDD patients; wherein the biomarker is M6PR, VPS13D, TNFRSF1A, PAPOLA, EFS, AGO2, ATE1, AFM, ENO2, EXOC2, GCA, ITGB1BP1, ATAD2B, ACHE, INSIG2, GNG11, TUBGCP2, NT5E, TTYH3, TANK, ATF7IP, CORO1C, LPCAT3, CERT1, ARL1, ATP5F1B, KCNH2, YY1, PIK3R3, LUC7L2, STAM2, AGPS, HERC5, PDCD6, ENPEP, RBM27, PIP5K1B, BLVRA, TCIRG1, SLC18A1, RBM6, TNK1, LGR5, RB1, RHOF, CD53, UBR3, ISY1, USP53, MARCHF1, MARCHF6, DAAM2, MAT2B, PPP1R21, GLT1D1, KIF5A, ANKRD9, BUB1B, WIPI2, JAML, PSMC2, TPCN2, STPG2, TMEM169, PPP1CB, ADH5, CAMKV, TTC39B, STK32C, DPP8, STEAP4, HNRNPR, SLC6A19, LDB2, CHSY3, TSPAN5, FSTL5, PRKCE, PAIP1, RPL15, DDX23, DHX36, ZNF622, ABLIM3, MAL, TADA2B, SLC39A1, GLUL, LSP1, DST, VWC2L, H2BC4, ANO1, PHLN1, CDHR3, ZFYVE1, PTPN6, HTR1F, TAGLN2, ZBTB38, ZNF513, DTX2, CUL1, RABGAP1L, SCMH1, MIPOL1, BTNL9, COA5, EEF1G, CABCOCO1, MRTO4, CD8B, SLC6A17, CEP63, WSCD2, TSKU, TESK1, DHX15, FGD3, ZNF385A, TMEM167B, CPM, RIMS2, ADIPOR1, ATP2B4, HSL1, OAS2, CCDC18, NKX2-3, LPAR6, ATG16L1, SCEL, C7orf25, SPATA16, PPTC7, PRPF40A, ARNT, FAR1, CACNA2D1, KPNA5, PPP4R2, ELAVL4, RC3H2, NFIA, TNKS1BP1, ECHDC2, TXNDC15, MARCHF5, GULP1, PIK3CG, LRP10, SLC39A10, RPL37A, UBAP2, NCKAP1, CLSTN1, Y_RNA, RNU5B-1, RNU6-1309P, RYR2, LGALS8, DNAH14, TMEM181, MDM4, TOR1AIP2, IFNGR1, HBS1L, TMCO1, SELENBP1, PDSS2, EIF3A, PNISR, CD58, DUSP5, SORCS3, SUFU, PRMT6, LRRC40, ADGRF4, FUT11, ANXA7, HDAC1, PHYHIPL, FGR, FBXO42, ZBTB17, TMEM51, GPR183, ATG2A, ST8SIA6, OR5L2, ESD, API5, B3GALT6, SLC7A1, VGLL3, CADM2, RNU6-1062P, RNU6-629P, SNORD26, RNU6-43P, SNORD64, RYR3, IGKV3-15, CCDC50, NUDT14, ARHGAP9, SYNE2, LBH, RASGEF1A, DDHD2, GNAT3, CLU, RPS6KA2, KMT5B, ARHGAP25, FAT3, MXD1, RNU2-29P, RNU2-59P, CROCCP2, NBEAL2, KBTBD8, ISY1-RAB43, ACTG1P19, HTATIP2, THEM7P, ATF7, BSN-DT, TMEM132D, LINC-PINT, INO80D, LINC00993, MTCO1P51, SOX5, AHCYP2, IRAK4, LINC01807, LRRC23, ANXA2P2, FHIP1A, ABTB2, TRABD2B, TAX1BP1-AS1, TRGV9, POTEI, CUL3, HRAT17, LINC02540, CFLAR, RNU7-1, SNORD121B, RNU7-19P, NDUFA5, CAP1, TRA2B, EIF3B, CTBP2, GNA12, ADGRL2, FRMD4A, TP53BP1, CYB5R4, AOAH, ENPP7P4, UBE4B, RHOBTB1, RN7SL242P, HNRNPA2BI, PLA2G4E-AS1, LIFR-AS1, COPB2, DSTNP2, PDCD6-AHRR, CTBP1-DT, DDX60L, ZDHHC11B, NREP, FAF2, FHDC1, SCARNA5, RNU6-1183P, PABPC1, SLA, SLC45A4, SOX6, IL10RA, DLG2, KMT2A, E2F8, XKR6, GLYATL2, SNHG1, LINC02376, C12orf57, FMC1-LUC7L2, GPHN, LINC02416, SNHG14, ACTN1, LINC00639, CCDC85C, MARK3, LINC02533, ATF7-NPFF, RNU6-84P, H4C1, WASF2, PWAR5, PLCL1, MAMDC2-AS1, LINC01170, SYNGAP1-AS1, LINC00862, PHGDH, LINC02752, LINC01117 or PRKN.

17. The biomarker according to claim 4, wherein the biomarker is RNU2-51P or PGAP1.

18. The biomarker according to claim 5, wherein the biomarker is used for distinguishing HC and PD patients, and the PD patients are PDND patients; wherein the biomarker is NRSN1, APPL2, TNFAIP8, ATP11A, LINC00847, CASP10, SNX16, MTA3, GRAPL, SMARCA2, LYPD3, ZNF252P-AS1, IRF2, TM9SF2, UNC79, LDC1P, GPR180, EFCAB5, PARP16, SH3D19, VPS37A, THAP8, IQSEC2, PTPRD, WDR7, INO80D, POLR1B, CCDC85C, CPE, STK24, DAAM2-AS1, CTSZ, DNAJC9, HSDL2-AS1, CBR4, IDI2-AS1, ACO2, ADNP-AS1, DIP2C-AS1, LINC00624, MAX, SRGAP2-AS1, NMT1, KLC4, KMT2E, LINC00506, ZNF565, ERAP1, TICAM2-AS1, PIGH, UFL1-AS1, VIM-AS1, KCNQ1OT1, TCHP, RB1, LIX1L-AS1, KCNMA1-AS3, PIGC, CYB5D2, FAM184B, FAR2, CRLF3, VCAN-AS1, STRADB, FTX, SKP2, RBFOX1, RPTOR or GNG12-AS1.

19. The biomarker according to claim 5, wherein the biomarker is used for distinguishing HC and PD patients, and the PD patients are PDD patients; wherein the biomarker is NRSN1, SGMS1-AS1, IRF2, LINC00847, SMARCA2, WDR87BP, LYPD3, ATP11A, APPL2, TM9SF2, GPR180, PREPL, YJU2, TNFAIP8, MT-CO3, FAT3, MTCO3P12, SH3D19, MT-TH, IQSEC2, UNC79, CCDC85C, LBHD1, SLC5A2, ARG2, GPR156, AUNIP, FOXM1, IDI2-AS1, TMEM165 or C1QTNF7-AS1.

20. The biomarker according to claim 5, wherein the biomarker is used for distinguishing PDND and PDD patients; wherein the biomarker is NTAQ1, SPINT2, KIF27, THSD1, SMU1, MT-ND3 or MEF2A.

21. The biomarker according to claim 5, wherein the biomarker is MT-CO3, SH3D19, TM9SF2, LYPD3, LINC00847, PREPL, YJU2, GPR180, IRF2, IQSEC2, ATP11A, GRAPL, CASP10, UNC79, SMARCA2, ZNF252P-AS1 or MTA3.

22. The biomarker according to claim 1, wherein the piRNA targets chromosome 1, 5, 7, 14, 15, 16, 17, 18 or 19 of the brain.

23. The biomarker according to claim 22, wherein the piRNA targets chromosome 2, 6, 9, 11, 13, 15, 16 or 17 of the brain.

24. A method for detecting whether an individual has Parkinson's disease, comprising the following steps:
I. acquiring a specimen from the individual;
II. separating a target detection object from the specimen;
III. analyzing the expression quantity of the target detection object;
IV. comparing to find out whether the target detection object contains the biomarker according to any one of claims 1 to 23; and
V. comparing to find a symptom of Parkinson's disease corresponding to the biomarker.

25. The detection method according to claim 24 wherein the specimen is blood or a brain tissue.

26. A kit comprising a reagent of the biomarker according to any one of claims 1 to 23.
